(19) United States
(12) Patent Application Publication
(10) Pub. No.: US 2026/0366063 A1
(43) Pub. Date: Nov. 19, 2026

(54) SUPER-RESOLUTION MICROSCOPY (SRM) MULTIPLEXING WITH CLEAVABLE MOIETIES

(71) Applicant: Toreador Therapeutics, Inc., San Francisco, CA (US)

(72) Inventors: Jeanmarie GUENOT, San Francisco, CA (US); Klaus Fiebig, Toronto (CA)

(21) Appl. No.: 19/153,719

(22) PCT Filed: Feb. 7, 2024
(86) PCT No.: PCT/US2024/014894

§ 371 Date: Aug. 5, 2025

Related U.S. Application Data
(60) Provisional application No. 63/483,916, filed on Feb. 8, 2023.

(51) Int. Cl. C12Q 1/6823; C12Q 1/6804; G01N 33/53

(52) U.S. Cl. CPC C12Q 1/6823; C12Q 1/6804; G01N 33/5308; G01N 2458/10

(57) ABSTRACT

Described herein are methods of providing a super-resolution microscopy (SRM) image of a target molecule expressed on cell surfaces of a cell sample. Further disclosed herein are methods of providing multiplexed SRM images of a number of different target molecules expressed on cell surfaces of a cell sample.

### CROSS-REFERENCE

The present application claims the benefit of U.S. Provisional Application No. 63/483,916 filed on Feb. 8, 2023 which is incorporated herein by reference in its entirety.

### SUMMARY

In an aspect, the present disclosure provides methods of providing a super-resolution microscopy (SRM) image of a target molecule expressed on cell surfaces of a cell sample. In some embodiments, a method of the present disclosure comprises: a) labeling the target molecule with a docking nucleic acid strand; b) hybridizing an imaging nucleic acid strand to the docking nucleic acid strand, wherein the imaging nucleic acid strand includes a fluorophore and at least one cleavable moiety, and wherein the hybridizing brings the fluorophore in proximity to the target molecule; c) imaging the cell sample to provide the SRM image of the target molecule; and d) cleaving the cleavable moiety to displace the fluorophore from proximity to the target molecule, to change a blinking behavior of the imaging nucleic acid strand, and/or to change a binding specificity of the imaging nucleic acid strand. In some embodiments, cleaving the cleavable moiety displaces the fluorophore from proximity to the target molecule.

In some embodiments, the method further comprises providing a multiplexed image of a number (n) of different target molecules expressed on the cell surfaces of the cell sample by: repeating steps a) to d) for each of the n different target molecules to provide an SRM image of each of the n different target molecules; and combining the SRM images of the n different target molecules to provide the multiplexed image.

In an aspect, the present disclosure provides methods of providing a multiplexed super-resolution microscopy (SRM) image of a number (n) of different target molecules expressed on cell surfaces of a cell sample. In some embodiments, a method of the present disclosure comprises: i) labeling each of the different target molecules with a different docking nucleic acid strand; ii) obtaining a SRM image of at least one target molecule of the n different target molecules by: hybridizing an imaging nucleic acid strand to the docking nucleic acid strand associated with the target molecule, wherein the imaging nucleic acid strand includes a fluorophore and a cleavable moiety, and wherein the hybridizing brings the fluorophore of the imaging nucleic acid strand in proximity to the target molecule, imaging the cell sample using fluorescence emission of the fluorophore, and cleaving the cleavable moiety to remove the fluorophore from proximity to the target molecule, to change a blinking behavior of the imaging nucleic acid strand, and/or to change a binding specificity of the imaging nucleic acid strand; iii) repeating step ii) until SRM images of all of the n different target molecules of the cell sample are obtained; and iv) combining the SRM images of all of the n different target molecules to provide the multiplexed SRM image of the n different target molecules expressed on the cell surfaces of the cell sample.

In some embodiments, imaging the cell sample comprises imaging the cell sample by stimulated emission depletion (STED) microscopy. In some embodiments, a different imaging strand is used for imaging each of the n different target molecules. In some embodiments, one imaging nucleic acid strand is used to image a subset of two or more of the n different target molecules. In some embodiments, the step of ii) obtaining the SRM image comprises obtaining an SRM image of a subset of target molecules of the n different target molecules simultaneously. In some embodiments, a different imaging nucleic acid strand with a different fluorophore is used for imaging each of the target molecules in the subset. In some embodiments, the subset includes at least 2 different target molecules. In some embodiments, the subset includes at least 3 different target molecules.

In some embodiments, the number (n) of different target molecules is at least 10. In some embodiments, n is at least 20. In some embodiments, n is at least 50. In some embodiments, n is at least 100.

In some embodiments, cleaving the cleavable moiety comprises cleaving the cleavable moiety with light. In some embodiments, cleaving the cleavable moiety comprises cleaving the cleavable moiety with UV light. In some embodiments, cleaving the cleavable moiety comprises cleaving the cleavable moiety via chemical cleavage. In some embodiments, cleaving the cleavable moiety comprises cleaving the cleavable moiety via enzymatic cleavage. In some embodiments, cleaving the cleavable moiety comprises cleaving the cleavable moiety with a nuclease.

In some embodiments, cleaving the cleavable moiety releases the fluorophore from the imaging nucleic acid strand. In some embodiments, cleaving the cleavable moiety releases at least a portion of the imaging nucleic acid strand from the docking nucleic acid strand. In some embodiments, the method further comprises displacing the imaging nucleic acid strand from the docking nucleic acid strand by hybridizing a non-labeled nucleic acid strand to the docking nucleic acid strand after cleaving the cleavable moiety. In some embodiments, cleaving the cleavable moiety releases the imaging nucleic acid strand from the docking nucleic acid strand. In some embodiments, cleaving the cleavable moiety changes kinetics of dissociation of the imaging nucleic acid strand from the docking nucleic acid strand, thereby changing the blinking behavior of the imaging nucleic acid strand. In some embodiments, cleaving the cleavable moiety changes the binding specificity of the imaging nucleic acid strand for the docking nucleic acid strand.

In some embodiments, the cleavable moiety is located between the fluorophore and the imaging nucleic acid strand. In some embodiments, the cleavable moiety is located within the sequence of the imaging nucleic acid strand. In some embodiments, the cleavable moiety is located between nucleotides of the imaging nucleic acid strand. In some embodiments, the imaging nucleic acid strand includes at least two cleavable moieties. In some embodiments, the imaging nucleic acid strand includes at least two cleavable moieties that are independently cleavable.

In some embodiments, the hybridizing brings the fluorophore within a distance of about 2 nanometers (nm) to about 10 nm from the target molecule. In some embodiments, the fluorophore is located at the 5′-end of the imaging nucleic acid strand. In some embodiments, the fluorophore is located at the 3′-end of the imaging nucleic acid strand. In some embodiments, the fluorophore is located between the 5′-end and the 3′-end of the imaging nucleic acid strand. In some embodiments, the fluorophore is located at or near a middle of the imaging nucleic acid strand.

In some embodiments, the imaging nucleic acid strand comprises deoxyribonucleic acid (DNA). In some embodiments, the imaging nucleic acid strand comprises peptide nucleic acid (PNA). In some embodiments, the docking nucleic acid strand comprises DNA. In some embodiments, the docking nucleic acid strand comprises PNA. In some embodiments, one or both of the docking nucleic acid strand and the imaging nucleic acid strand comprise ribonucleic acid (RNA). In some embodiments, one or both of the docking nucleic acid strand and the imaging nucleic acid strand comprise an aptamer. In some embodiments, one or both of the docking nucleic acid strand and the imaging nucleic acid strand comprise one or more non-natural nucleic acids. In some embodiments, the one or more non-natural nucleic acids comprise one or more phosphorothionate backbone linkages. In some embodiments, the one or more non-natural nucleic acids comprise one or more boranophosphate backbone linkages. In some embodiments, the one or more non-natural nucleic acids comprise one or more phosphonate backbone linkages. In some embodiments, the one or more non-natural nucleic acids comprise threose nucleic acid (TNA). In some embodiments, the one or more non-natural nucleic acids comprise locked nucleic acid (LNA). In some embodiments, the one or more non-natural nucleic acids comprise hexitol nucleic acid (HNA). In some embodiments, the one or more non-natural nucleic acids comprise flexible nucleic acid (FNA). In some embodiments, the one or more non-natural nucleic acids comprise glycerol nucleic acid (GNA). In some embodiments, the one or more non-natural nucleic acids comprise a nucleobase analog.

In some embodiments, the cell sample comprises disseminated cells. In some embodiments, the cell sample comprises tissue fragments. In some embodiments, the cell sample comprises a tissue sample. In some embodiments, the tissue sample is fixed. In some embodiments, the method further comprises fixing the cell sample. In some embodiments, the tissue sample is a live tissue sample. In some embodiments, the cell sample comprises cultured cells. In some embodiments, the cultured cells are fixed. In some embodiments, the cultured cells are live.

In some embodiments, the method is carried out without changing buffer conditions of the cell sample. In some embodiments, all of the steps of the method are carried out in a same buffer. In some embodiments, the method is carried out in physiological buffer conditions. In some embodiments, all of the steps of the method are carried out in a single buffer having a physiological pH and a physiological salt concentration. In some embodiments, all of the steps of the method are carried out in a single buffer having a pH in a range of about 7.2 to about 7.5, and a sodium chloride concentration in a range of about 135 millimolar (mM) to about 145 mM. In some embodiments, the method is carried out in a buffer having a sodium chloride concentration in a range of about 135 millimolar (mM) to about 145 mM. In some embodiments, all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4. In some embodiments, one or more steps of the method are carried out in a buffer having a salt concentration of about 500 millimolar (mM). In some embodiments, one or more of the steps of the method are carried out in a buffer having a salt concentration in a range of about 500 millimolar (mM) to about 600 mM. In some embodiments, one or more steps of the method are carried out in a buffer having a salt concentration of about 50 millimolar (mM).

In some embodiments, the cleaving removes the fluorophore from the imaging nucleic acid strand. In some embodiments, the method further comprises washing the cell sample after the cleaving. In some embodiments, the method further comprises acquiring an SRM or confocal image of the cell sample after the washing to confirm removal of the fluorophore.

In some embodiments, the cleaving causes the imaging nucleic acid strand to dissociate from the docking nucleic acid strand. In some embodiments, the cleaving causes the imaging nucleic acid strand to expose a different sequence of nucleotide that binds to a different docking nucleic acid strand. In some embodiments, the cleaving removes the fluorophore and at least a portion of the imaging nucleic acid strand from the docking nucleic acid strand. In some embodiments, the method further comprises washing the cell sample after the cleaving. In some embodiments, the method further comprises acquiring an SRM or confocal image of the cell sample after the washing to confirm removal of the fluorophore and the imaging nucleic acid strand from the docking nucleic acid strand.

In some embodiments, the hybridizing results in a stable complex being formed between imaging nucleic acid strand and the docking nucleic acid strand. In some embodiments, the stable complex has a lifetime of at least about 1 second. In some embodiments, the stable complex has a lifetime of at least about 1 hour. In some embodiments, the stable complex has a lifetime of more than 1 hour. In some embodiments, the imaging nucleic acid strand exhibits non-blinking behavior due to the stable complex formed between the imaging nucleic acid strand and the docking nucleic acid strand. In some embodiments, the cleaving destabilizes the stable complex formed between the imaging nucleic acid strand and the docking nucleic acid strand, thereby causing the imaging nucleic acid strand and the docking nucleic acid strand to form a transient complex, and causing the imaging nucleic acid strand to exhibit blinking behavior. In some embodiments, the cleaving causes the imaging strand to exhibit a blinking behavior.

In some embodiments, the hybridizing results in a transient complex being formed between the imaging nucleic acid strand and the docking nucleic acid strand. In some embodiments, the imaging nucleic acid strand exhibits blinking behavior due to the transient complex formed between the imaging nucleic acid strand and the docking nucleic acid strand. In some embodiments, the cleaving modulates binding properties of the transient complex, resulting in a change in frequency and/or duration of blinking of the imaging nucleic acid strand. In some embodiments, the cleaving modulates binding properties of the transient complex, causing the imaging nucleic acid strand to bind to a docking nucleic acid strand of another target molecule. In some embodiments, kinetics of the blinking behavior is correlated with a concentration of the imaging nucleic acid strand.

In some embodiments, the target molecule comprises a cancer protein. In some embodiments, the target molecule comprises a tumor antigen. In some embodiments, the target molecule comprises a ligand. In some embodiments, the target molecule comprises a lipid. In some embodiments, the target molecule comprises a carbohydrate.

In some embodiments, labeling the target molecule comprises directly connecting the docking nucleic acid strand to the target molecule. In some embodiments, labeling the target molecule comprises binding a binding molecule to the target molecule, wherein the binding molecule selectively binds to the target molecule, and wherein the binding molecule is connected to the docking nucleic acid strand. In some embodiments, labeling the target molecule comprises connecting the docking nucleic acid strand to the binding molecule by a linker.

In some embodiments, the binding molecule is selected from an antibody, a vhh antibody, a single-chain variable fragment (scFv), a Fab antibody fragment, a Fab antibody, a secondary antibody, a ligand, and a cofactor. In some embodiments, the ligand comprises a small molecule or a protein. In some embodiments, the binding molecule is a vhh antibody.

In some embodiments, the linker comprises a polypeptide. In some embodiments, the linker connects the N-terminus or C-terminus of the binding molecule with the 5′-end or the 3′-end of the docking nucleic acid strand. In some embodiments, the binding molecule and the docking nucleic acid strand are connected via a sortase-mediated ligation. In some embodiments, the sortase-mediated ligation comprises ligating a C-terminal LPXTG-XX sequence of the binding molecule with a GGG peptide at the 5′-end of the docking nucleic acid strand. In some embodiments, the binding molecule is connected to the docking nucleic acid strand via a side chain of the binding molecule. In some embodiments, the side chain is an arginine side chain or a cysteine side chain.

In some embodiments, the present disclosure provides cell samples for super-resolution microscopy (SRM) imaging. In some embodiments, a cell sample of the present disclosure comprises a target molecule expressed on cell surfaces of the cell sample, a docking nucleic acid associated with the target molecule, and an imaging nucleic acid strand hybridized to the docking nucleic acid strand, wherein the imaging nucleic acid strand includes a fluorophore and a cleavable moiety.

In some embodiments, the cleavable moiety is cleavable with light. In some embodiments, the cleavable moiety is cleavable with UV light. In some embodiments, the cleavable moiety is cleavable by chemical cleavage. In some embodiments, the cleavable moiety is cleavable by enzymatic cleavage. In some embodiments, the cleavable moiety is cleavable with a nuclease. In some embodiments, the cleavable moiety is located between the fluorophore and the imaging nucleic acid strand. In some embodiments, the cleavable moiety is located within the sequence of the imaging nucleic acid strand. In some embodiments, the cleavable moiety is located between nucleotides of the imaging nucleic acid strand. In some embodiments, the imaging nucleic acid strand includes at least two cleavable moieties. In some embodiments, the at least two cleavable moieties are independently cleavable.

In some embodiments, the fluorophore is located at the 5′-end of the imaging nucleic acid strand. In some embodiments, the fluorophore is located at the 3′-end of the imaging nucleic acid strand. In some embodiments, the fluorophore is located between the 5′-end and the 3′-end of the imaging nucleic acid strand. In some embodiments, the fluorophore is located at or near a middle of the imaging nucleic acid strand.

In some embodiments, the imaging nucleic acid strand comprises DNA. In some embodiments, the imaging nucleic acid strand comprises PNA. In some embodiments, the docking nucleic acid strand comprises DNA. In some embodiments, the docking nucleic acid strand comprises PNA. In some embodiments, one or both of the docking nucleic acid strand and the imaging nucleic acid strand comprise RNA. In some embodiments, one or both of the docking nucleic acid strand and the imaging nucleic acid strand comprise an aptamer. In some embodiments, one or both of the docking nucleic acid strand and the imaging nucleic acid strand comprise one or more non-natural nucleic acids. In some embodiments, the one or more non-natural nucleic acids comprise one or more phosphorothionate linkages. In some embodiments, the one or more non-natural nucleic acids comprise one or more phosphorothionate linkages. In some embodiments, the one or more non-natural nucleic acids comprise one or more boranophosphate linkages. In some embodiments, the one or more non-natural nucleic acids comprise one or more phosphonate linkages. In some embodiments, the one or more non-natural nucleic acids comprise threose nucleic acid (TNA). In some embodiments, the one or more non-natural nucleic acids comprise locked nucleic acid (LNA). In some embodiments, the one or more non-natural nucleic acids comprise hexitol nucleic acid (HNA). In some embodiments, the one or more non-natural nucleic acids comprise flexible nucleic acid (FNA). In some embodiments, the one or more non-natural nucleic acids comprise glycerol nucleic acid (GNA). In some embodiments, the one or more non-natural nucleic acids comprise a nucleobase analog.

In some embodiments, the cell sample comprises a tissue sample. In some embodiments, the tissue sample is fixed. In some embodiments, the tissue sample is a live tissue sample. In some embodiments, cell sample comprises cultured cells. In some embodiments, the cultured cells are fixed. In some embodiments, the cultured cells are live. In some embodiments, the cell sample comprises disseminated cells. In some embodiments, the cell sample comprises tissue fragments.

In some embodiments, the docking nucleic acid strand and the imaging nucleic acid strand form a stable complex. In some embodiments, the stable complex has a lifetime of at least about 1 second. In some embodiments, the stable complex has a lifetime of at least about 1 hour. In some embodiments, the stable complex has a lifetime of more than 1 hour.

In some embodiments, the target molecule comprises a cancer protein. In some embodiments, the target molecule comprises a tumor antigen. In some embodiments, the target molecule comprises a ligand. In some embodiments, the target molecule comprises a lipid. In some embodiments, the target molecule comprises a carbohydrate.

In some embodiments, the target molecule is directly connected to the docking nucleic acid strand. In some embodiments, the docking nucleic acid strand is connected to a binding molecule by a linker, and the binding molecule is selectively bound to the target molecule. In some embodiments, the binding molecule is selected from an antibody, a vhh antibody, a single-chain variable fragment (scFv), a Fab antibody fragment, a Fab antibody, a secondary antibody, a ligand, and a cofactor. In some embodiments, the ligand comprises a small molecule or a protein. In some embodiments, the binding molecule is a vhh antibody.

In some embodiments, the linker comprises a polypeptide. In some embodiments, the binding molecule is connected to the docking nucleic acid strand via a side chain of the binding molecule. In some embodiments, the side chain is an arginine side chain or a cysteine side chain.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, of which:

FIG. 1 shows a schematic representation of a strategy for super-resolution microscopy (SRM) imaging of a target molecule expressed on a cell surface of a cell sample in accordance with the present disclosure. An imaging nucleic acid strand carrying a fluorophore is hybridized to a docking nucleic acid strand associated with the target molecule. A cleavable moiety in the imaging nucleic acid strand is cleaved following SRM imaging.

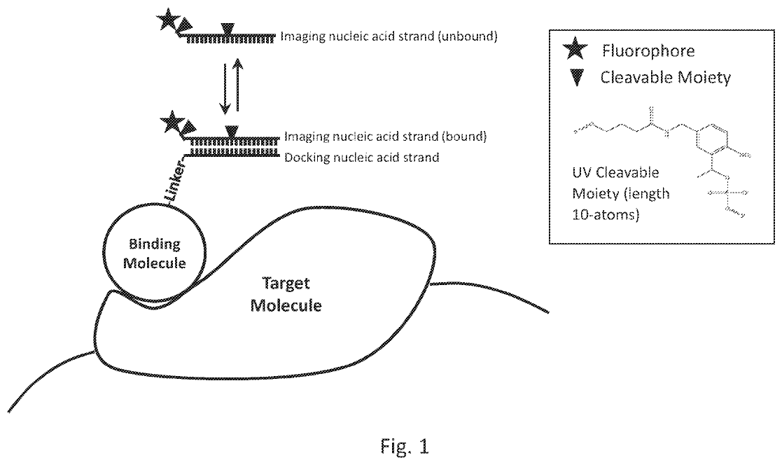

FIG. 2 shows an example of a strategy for SRM multiplex imaging of n different target molecules expressed on cell surfaces of a cell sample in accordance with the present disclosure.

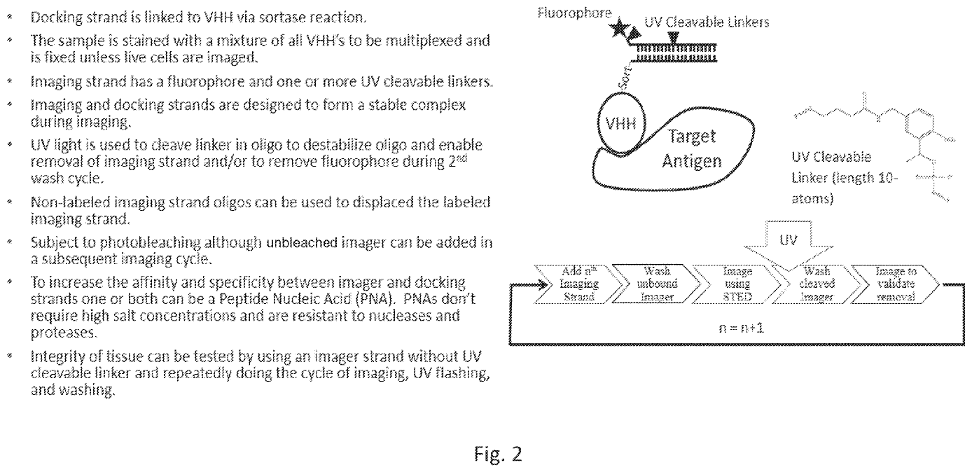

FIG. 3 shows an exemplary cleavable moiety in accordance with the present disclosure.

FIG. 4 is a flowchart of an exemplary method of SRM multiplex imaging of n different target molecules expressed on cell surfaces of a cell sample using a fluorescently-labeled imaging strand having a cleavable moiety in accordance with the present disclosure. The method allows for sequential multiplex imaging of a large number (n) of different target molecules via sequential hybridization, imaging, cleaving, and wash cycles.

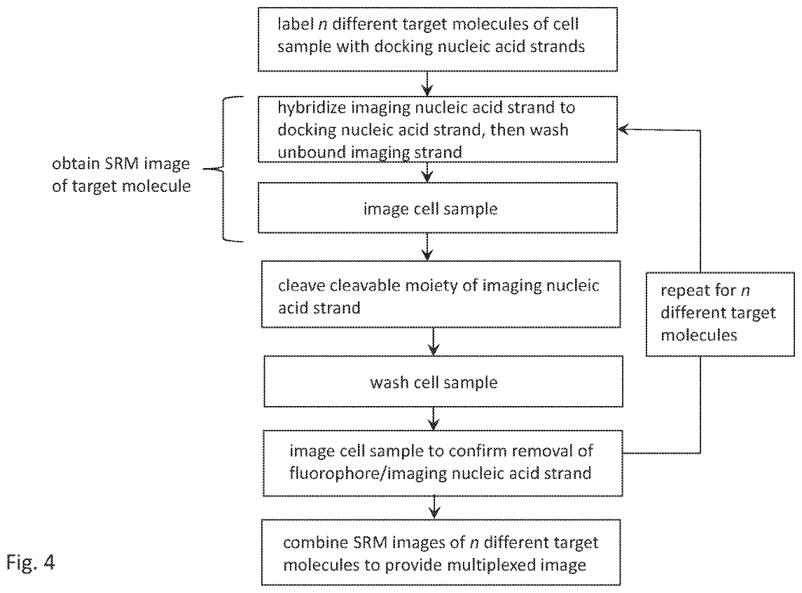

FIG. 5 shows an example of sortase-mediated ligation for linking a binding molecule to a docking nucleic acid strand.

FIG. 6 shows a schematic representation of a strategy for SRM imaging of a target molecule expressed on a cell surface of a cell sample in accordance with the present disclosure. An imaging nucleic acid strand carrying a fluorophore and a cleavable moiety is bound to the target molecule. The cleavable moiety is cleaved following SRM imaging.

FIG. 7 shows a schematic representation of a strategy for SRM imaging of a target molecule expressed on a surface of a cell sample in accordance with the present disclosure. A binding molecule that binds to the target molecule is connected directly or via a linker to a cleavable moiety and a fluorophore. The cleavable moiety is cleaved following SRM imaging.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

As used herein, a “cell sample” includes a cell sample or a tissue sample. In non-limiting embodiments, the cell sample or tissue sample may be from a human or an animal (e.g., cynomolgus monkey). Non-limiting examples of cell samples include cancer cells, normal or non-cancerous cells, nerve cells, muscle cells, connective tissue cells, and blast cells (e.g., myeloid blast cells).

### DEFINITIONS

Super-resolution microscopy (SRM) is an optical microscopy technique which allows images to have resolutions higher than those imposed by the diffraction limit. Stimulated emission depletion (STED) is a type of SRM fluorescence microscopy technique which overcomes the diffraction limit of confocal microscopes. The resolution enhancement is based on switching off the fluorescence of dye molecules by stimulated emission using intense laser light in the outer regions of the diffraction limited excitation focus. This intense radiation causes almost all of the excited molecules to return to the ground state. Fluorescence from the remaining excited dye molecules in the center of the excitation focus is then detected and used to form the high resolution images.

A “multiplexed image” or “multiplexed SRM image” as used herein is an image combining the SRM imaging results of different target molecules imaged using multiple fluorescent dyes. “SRM multiplexing” as used herein refers to the detection of multiple targets by SRM using multiple fluorescent dyes.

“Blinking behavior” as used herein is a property of the imaging nucleic acid strand that carries the fluorophore, and refers to whether or not a fluorescent signal of the fluorophore appears to flicker on and off (or blink) with SRM imaging. “Blinking behavior” also refers to the properties of the blinking of the imaging nucleic acid strand including a frequency and/or duration of the blinking. Blinking behavior may be correlated with the stability of the complex formed between the imaging nucleic acid strand and the docking nucleic acid strand, with more stable complexes exhibiting less blinking and more transient complexes exhibiting more blinking. An imaging nucleic acid strand that is “blinking” refers to an imaging nucleic acid strand in which the fluorescent signal associated with its fluorophore appears to flicker on and off with SRM imaging. An imaging nucleic acid strand that is “non-blinking” refers to an imaging nucleic acid strand in which the fluorescent signal associated with its fluorophore appears constant and does not flicker on and off.

The terms “polypeptide” and “protein” are used interchangeably to refer to a polymer of amino acid residues, and are not limited to a minimum length. Polypeptides, including the provided polypeptides and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

In some embodiments, amino acid sequence variants of the polypeptides provided herein are contemplated. A variant typically differs from a polypeptide specifically disclosed herein in one or more substitutions, deletions, additions and/or insertions. Such variants can be naturally occurring or can be synthetically generated. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

As used herein, the term “nucleic acid” refers to a biopolymer composed of monomer units each having a nucleobase (e.g., cytosine, guanine, adenine, thymine, uracil). Nucleic acids as used herein may be single-stranded, double-stranded, or partially single-stranded and partially double-stranded. Nucleic acids may be structured (e.g., hairpin, aptamer) or may be unstructured. Exemplary nucleic acids include, but are not limited to, deoxyribonucleic acid (DNA), ribonucleic acid (RNA), and peptide nucleic acid (PNA). Also contemplated are natural and non-natural nucleic acids, including nucleic acids having non-natural backbones and/or non-natural nucleobase analogs.

As used herein, a “target molecule” refers to a molecule of interest expressed on cell surfaces of a cell sample. The target molecule may include, but is not limited to, a cancer protein or a cancer target molecule (e.g., a tumor antigen or protein or molecule involved in cancer or a cancer pathway), a ligand (e.g., a small molecule ligand or a protein ligand), a lipid, or a carbohydrate.

As used herein, a “binding molecule” is a molecule that binds to a target molecule with high specificity. For example, a binding molecule as used herein may comprise an antibody, a vhh antibody, a single-chain variable fragment (scFv), a Fab antibody fragment, a Fab antibody, a secondary antibody, a ligand, a cofactor, a nucleic acid, or any other molecule that binds to the target molecule with high specificity.

### SRM Imaging of Multiple Target Molecules Using Cleavable Moieties

The detection of multiple target molecules (multiplexing) at once in a biological sample may be desirable for characterization of the biological sample. For example, multiplexing may provide a map of an expression profile of a tissue sample, and may be used to distinguish the expression profiles of diseased (e.g., cancerous) tissue from normal tissue. Existing strategies for SRM multiplexing involve sequentially attaching, imaging, and removing dye-modified DNA strands from complementary strands that are coupled to different target molecules. Current methods use a change in salt concentration for controlling hybridization and removal of the dye-modified DNA strands, with a high salt concentration (e.g., 500 millimolar (mM) to 600 mM NaCl) promoting hybridization, and a low salt concentration (e.g., 50 mM NaCl) promoting removal. Chemical denaturants (e.g., formamide) may also be used for triggering removal of the dye-modified DNA strands. However, repeated switching of buffer conditions between high and low salt concentration and/or exposure to chemical denaturants could be damaging to live cell and tissue samples. There is a need for alternative SRM multiplexing strategies that are less damaging to live cells. There is also a need for strategies for controlling the binding specificity and the blinking behavior of the dye-modified DNA strands.

In an aspect, the present disclosure provides methods of providing a super-resolution microscopy (SRM) image of a target molecule expressed on cell surfaces of a cell sample. In some embodiments, a method of the present disclosure comprises: a) labeling the target molecule with a docking nucleic acid strand; b) hybridizing an imaging nucleic acid strand to the docking nucleic acid strand, wherein the imaging nucleic acid strand includes a fluorophore and at least one cleavable moiety, and wherein the hybridizing brings the fluorophore in proximity to the target molecule; c) imaging the cell sample to provide the SRM image of the target molecule; and d) cleaving the cleavable moiety to displace the fluorophore from proximity to the target molecule, to change a blinking behavior of the imaging nucleic acid strand, and/or to change a binding specificity of the imaging nucleic acid strand. In some embodiments, cleaving the cleavable moiety displaces the fluorophore from proximity to the target molecule. In some embodiments, cleaving the cleavable moiety changes a blinking behavior of the imaging nucleic acid strand. In some embodiments, cleaving the cleavable moiety changes a binding specificity of the imaging nucleic acid strand for the docking nucleic acid strand. In some embodiments, the method further comprises providing a multiplexed image of a number (n) of different target molecules expressed on the cell surfaces of the cell sample by repeating steps a) to d) for each of the n different target molecules to provide an SRM image of each of the n different target molecules, and combining the SRM images of the n different target molecules to provide the multiplexed image.

In an aspect, the present disclosure provides methods of providing a multiplexed super-resolution microscopy (SRM) image of a number (n) of different target molecules expressed on cell surfaces of a cell sample. In some embodiments, a method of the present disclosure comprises: i) labeling each of the different target molecules with a different docking nucleic acid strand; ii) obtaining a SRM image of at least one target molecule of the n different target molecules by: hybridizing an imaging nucleic acid strand to the docking nucleic acid strand associated with the target molecule, wherein the imaging nucleic acid strand includes a fluorophore and a cleavable moiety, and wherein the hybridizing brings the fluorophore of the imaging nucleic acid strand in proximity to the target molecule, imaging the cell sample using fluorescence emission of the fluorophore, and cleaving the cleavable moiety to remove the fluorophore from proximity to the target molecule, to change a blinking behavior of the imaging nucleic acid strand, and/or to change a binding specificity of the imaging nucleic acid strand; and iii) repeating step ii) until SRM images of all of the n different target molecules of the cell sample are obtained; and iv) combining the SRM images of all of the n different target molecules to provide the multiplexed SRM image of the n different target molecules expressed on the cell surfaces of the cell sample. In some embodiments, cleaving the cleavable moiety removes the fluorophore from proximity to the target molecule. In some embodiments, cleaving the cleavable moiety changes a blinking behavior of the imaging nucleic acid strand. In some embodiments, cleaving the cleavable moiety changes a binding specificity of the imaging nucleic acid strand for the docking nucleic acid strand.

In some embodiments, imaging the cell sample comprises imaging the cell sample by stimulated emission depletion (STED) microscopy. In some embodiments, imaging the cell sample comprises imaging the cell sample by structured illumination microscopy (SIM). In some embodiments, imaging the cell sample comprises imaging the cell sample by stochastic optical reconstruction microscopy (STORM)/photoactivation localization microscopy (PALM).

In some embodiments, a different imaging nucleic acid strand is used for imaging each of the n different target molecules. In some embodiments, one imaging nucleic acid strand is used to image a subset of two or more of the n different target molecules.

In some embodiments, the step of ii) obtaining the SRM image comprises obtaining an SRM image of a subset of target molecules of the n different target molecules. In some embodiments, a different imaging nucleic acid strand with a different fluorophore is used for imaging each of the target molecules of the subset. In some embodiments, the subset includes at least 2 different target molecules. In some embodiments, the subset includes at least 3 different target molecules.

In some embodiments, the number (n) of different target molecules is at least 10. In some embodiments, n is at least 20. In some embodiments, n is at least 30. In some embodiments, n is at least 40. In some embodiments, n is at least 50. In some embodiments, n is at least 100. In some embodiments, n is more than 100. In some embodiments, n is more than 500. In some embodiments, n is more than 1000.

### Cleavable Moiety

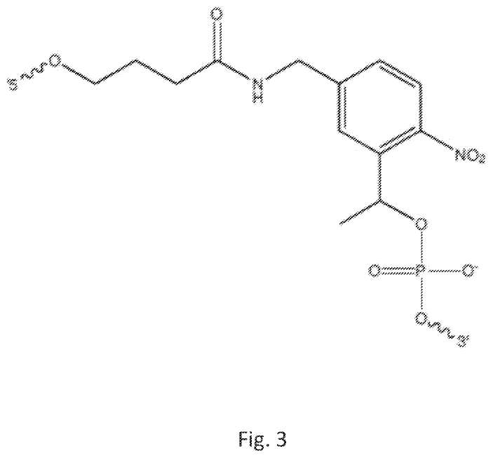

In some embodiments, cleaving the cleavable moiety comprises cleaving the cleavable moiety with light. In some embodiments, cleaving the cleavable moiety comprises cleaving the cleavable moiety with UV light. In some embodiments, the cleavable moiety has the structure shown in FIG. 3. In some embodiments, cleaving the cleavable moiety comprises cleaving the cleavable moiety with UV light having a wavelength in a range of about 300 nanometers (nm) to about 350 nm. In some embodiments, cleaving the cleavable moiety with light or UV light does not damage the cell sample.

In some embodiments, cleaving the cleavable moiety comprises cleaving the cleavable moiety via chemical cleavage. In some embodiments, cleaving the cleavable moiety comprises cleaving the cleavable moiety via enzymatic cleavage. In some embodiments, cleaving the cleavable moiety comprises cleaving the cleavable moiety with a nuclease.

In some embodiments, cleaving the cleavable moiety releases the fluorophore from the imaging nucleic acid strand. In some embodiments, cleaving the cleavable moiety releases at least a portion of the imaging nucleic acid strand from the docking nucleic acid strand. In some embodiments, the method further comprises displacing the imaging nucleic acid strand from the docking nucleic acid strand by hybridizing a non-labeled nucleic acid strand to the docking nucleic acid strand after cleaving the cleavable moiety. In some embodiments, cleaving the cleavable moiety releases the imaging nucleic acid strand from the docking nucleic acid strand. In some embodiments, cleaving the cleavable moiety changes kinetics of dissociation of the imaging nucleic acid strand from the docking nucleic acid strand, thereby changing the blinking behavior of the imaging nucleic acid strand. In some embodiments, cleaving the cleavable moiety changes the binding specificity of the imaging nucleic acid strand for the docking nucleic acid strand.

In some embodiments, the cleavable moiety is located between the fluorophore and the imaging nucleic acid strand. In some embodiments, the cleavable moiety is located at or near the 5′-end of the imaging nucleic acid strand. In some embodiments, the cleavable moiety is located at or near the 3′-end of the imaging nucleic acid strand. In some embodiments, the cleavable moiety is located between the 5′-end and the 3′-end of the imaging nucleic acid strand. In some embodiments, the cleavable moiety is located within the sequence of the imaging nucleic acid strand. In some embodiments, the cleavable moiety is located between nucleotides of the imaging nucleic acid strand. In some embodiments, the imaging nucleic acid strand includes at least two cleavable moieties. In some embodiments, the imaging nucleic acid strand includes at least two cleavable moieties that are independently cleavable.

### Fluorophore

In some embodiments, the hybridizing brings the fluorophore within a distance of about 2 nanometers (nm) to about 20 nm from the target molecule. In some embodiments, the hybridizing brings the fluorophore within a distance of about 2 nm to about 10 nm from the target molecule. In some embodiments, the hybridizing brings the fluorophore within a distance of about 2 nm to about 5 nm from the target molecule. In some embodiments, the hybridizing brings the fluorophore within a distance of about 2 nm from the target molecule.

In some embodiments, the fluorophore is located at the 5′-end of the imaging nucleic acid strand. In some embodiments, the fluorophore is located at the 3′-end of the imaging nucleic acid strand. In some embodiments, the fluorophore is located between the 5′-end and the 3′-end of the imaging nucleic acid strand. In some embodiments, the fluorophore is located at or near a middle of the imaging nucleic acid strand.

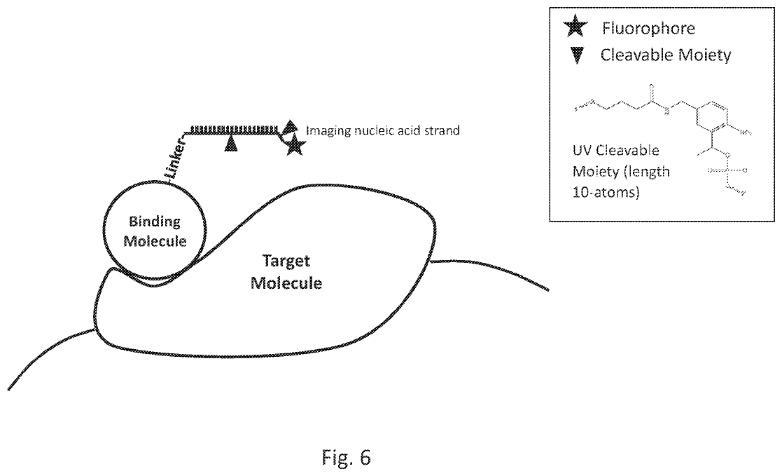

In some embodiments, the cleavable moiety and the fluorophore are incorporated within an imaging nucleic acid strand and a docking nucleic acid strand is not used (see FIG. 6). In such embodiments, the labeled binding molecule for each of the n different target molecules are added or flowed into the cell sample, imaged and cleaved sequentially. In some embodiments, the imaging nucleic acid strand carrying the cleavable moiety and the fluorophore is anchored to the target molecule via the binding molecule that binds specifically to the target molecule (see FIG. 6).

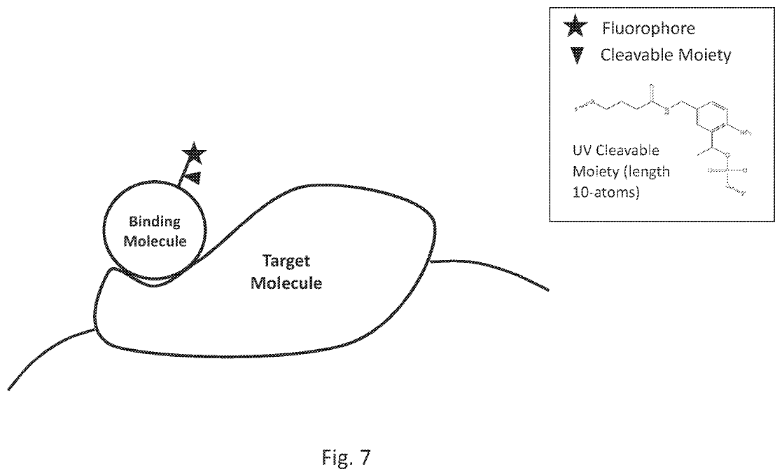

In some embodiments, the cleavable moiety and the fluorophore are connected directly or via a linker to the binding molecule without an imaging nucleic acid strand and a docking nucleic acid strand (see FIG. 7). In such embodiments, the binding molecule is not labeled with a docking nucleic acid strand. In such embodiments, the binding molecule for each of the n different target molecules are added or flowed into the cell sample, imaged and cleaved sequentially.

Non-limiting examples of a fluorophore includes, but are not limited to, abberior STAR RED, abberior STAR 635P, abberior STAR 635, ATTO 647N, ATTO 643, ATTO 633, abberior STAR ORANGE, abberior STAR 580, abberior STAR 600, ATTO 594, ATTO 590, abberior STAR GREEN, Oregon Green 488, abberior STAR 488, ATTO 488, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5-(or 6-) iodoacetamidofluorescein, 5-{[2 (and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, quantum dots, or other fluorophores.

### Nucleic Acid Strands

In some embodiments, the imaging nucleic acid strand and the docking nucleic acid strand are single-stranded nucleic acid strands. In some embodiments, the imaging nucleic acid strand and the docking nucleic acid strand are complementary to each other. In some embodiments, the imaging nucleic acid strand comprises deoxyribonucleic acid (DNA). In some embodiments, the imaging nucleic acid strand comprises peptide nucleic acid (PNA). In some embodiments, the docking nucleic acid strand comprises DNA. In some embodiments, the docking nucleic acid strand comprises PNA. In some embodiments, one or both of the imaging nucleic acid strand and the docking nucleic acid strand comprise PNA.

In some embodiments, one or both of the docking nucleic acid strand and the imaging nucleic acid strand comprise ribonucleic acid (RNA). In some embodiments, one or both of the docking nucleic acid strand and the imaging nucleic acid strand comprise an aptamer. In some embodiments, one or both of the docking nucleic acid strand and the imaging nucleic acid strand comprise one or more non-natural nucleic acids. In some embodiments, the one or more non-natural nucleic acids comprise a non-natural nucleic acid as described in Appella (Appella, D. H., “Non-natural Nucleic Acids for Synthetic Biology”, Curr. Opin. Chem. Biol. 2009, 13 (5-6), 687-696). In some embodiments, the one or more non-natural nucleic acids comprise one or more phosphorothionate backbone linkages. In some embodiments, the one or more non-natural nucleic acids comprise one or more boranophosphate backbone linkages. In some embodiments, the one or more non-natural nucleic acids comprise one or more phosphonate backbone linkages. In some embodiments, the one or more non-natural nucleic acids comprise threose nucleic acid (TNA). In some embodiments, the one or more non-natural nucleic acids comprise locked nucleic acid (LNA). In some embodiments, the one or more non-natural nucleic acids comprise hexitol nucleic acid (HNA). In some embodiments, the one or more non-natural nucleic acids comprise flexible nucleic acid (FNA). In some embodiments, the one or more non-natural nucleic acids comprise glycerol nucleic acid (GNA). In some embodiments, the one or more non-natural nucleic acids comprise a nucleobase analog.

### Cell Sample

In some embodiments, the cell sample comprises a tissue sample. In some embodiments, the tissue sample is fixed. In some embodiments, the tissue sample is a live tissue sample. In some embodiments, the cell sample comprises cultured cells. In some embodiments, the cultured cells are fixed. In some embodiments, the cultured cells are live. In some embodiments, the cell sample comprises disseminated cells. In some embodiments, the cell sample comprises tissue fragments. In some embodiments, the method further comprises fixing the cell sample prior to labeling the target molecule.

In some embodiments, the cell sample comprises cancer cells. In some embodiments, the cell sample comprises non-cancerous cells. In some embodiments, the cell sample comprises epithelial cells. In some embodiments, the cell sample comprises nerve cells. In some embodiments, the cell sample comprises muscle cells. In some embodiments, the cell sample comprises connective tissue cells. In some embodiments, the cell sample comprises normal (non-cancerous) or cancerous breast tissue cells. In some embodiments, the cell sample comprises normal or cancerous lung tissue cells. In some embodiments, the cell sample comprises blast cells (e.g., myeloid blast cells). In some embodiments, the cell sample comprises normal cells or cancer blast cells. In some embodiments, the cell sample includes hematologic cancer cells and/or their corresponding normal cells.

### Buffer Conditions

In some embodiments, the method is carried out without changing buffer conditions of the cell sample. In some embodiments, all of the steps of the method are carried out in a same buffer. In some embodiments, the method is carried out in physiological buffer conditions. In some embodiments, all of the steps of the method are carried out in a single buffer having a physiological pH and a physiological salt concentration. In some embodiments, all of the steps of the method are carried out in a single buffer having a pH in a range of about 7.2 to about 7.5, and a sodium chloride concentration in a range of about 135 millimolar (mM) to about 145 mM. In some embodiments, the method is carried out in a buffer having a sodium chloride concentration in a range of about 135 millimolar (mM) to about 145 mM. In some embodiments, all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4.

In some embodiments, one or more steps of the method are carried out in a buffer having a salt concentration of about 500 millimolar (mM). In some embodiments, one or more of the steps of the method are carried out in a buffer having a salt concentration in a range of about 500 millimolar (mM) to about 600 mM. In some embodiments, one or more steps of the method are carried out in a buffer having a salt concentration of about 50 millimolar (mM).

### Modification, Washing, and Validation

In some embodiments, the cleaving removes the fluorophore from the imaging nucleic acid strand. In some embodiments, the method further comprises washing the cell sample after the cleaving. In some embodiments, the method further comprises acquiring an SRM or confocal image of the cell sample after the washing to confirm removal of the fluorophore.

In some embodiments, the cleaving causes the imaging nucleic acid strand to dissociate from the docking nucleic acid strand. In some embodiments, the cleaving causes the imaging nucleic acid strand to expose a different sequence of nucleotide that binds to a different docking nucleic acid strand.

In some embodiments, the cleaving removes the fluorophore and at least a portion of the imaging nucleic acid strand from the docking nucleic acid strand. In some embodiments, the method further comprises washing the cell sample after the cleaving. In some embodiments, the method further comprises acquiring an SRM or confocal image of the cell sample after the washing to confirm removal of the fluorophore and the imaging nucleic acid strand from the docking nucleic acid strand.

### Stable Complex

In some embodiments, the hybridizing results in a stable complex being formed between imaging nucleic acid strand and the docking nucleic acid strand. In some embodiments, the stable complex has a lifetime of at least about 1 second. In some embodiments, the stable complex has a lifetime of at least about 1 hour. In some embodiments, the stable complex has a lifetime of more than 1 hour. In some embodiments, the imaging nucleic acid strand exhibits non-blinking behavior due to the stable complex formed between the imaging nucleic acid strand and the docking nucleic acid strand.

In some embodiments, the cleaving causes the imaging nucleic acid strand to exhibit a blinking behavior. In some embodiments, the cleaving results in a change in a blinking frequency and/or duration of the imaging nucleic acid strand. In some embodiments, the cleaving destabilizes the stable complex formed between the imaging nucleic acid strand and the docking nucleic acid strand, thereby causing the imaging nucleic acid strand and the docking nucleic acid strand to form a transient complex, and causing the imaging nucleic acid strand to exhibit blinking behavior. In some embodiments, the cleaving causes the blinking behavior of the imaging nucleic acid strand to shift from non-blinking to blinking.

In some embodiments, the imaging nucleic acid strand forms a stable complex with at least two different docking nucleic acid strands associated with at least two different target molecules, and the cleaving causes the imaging nucleic acid strand to form a transient complex with the at least two different docking nucleic acid strands with stronger binding to one of the two different docking nucleic acid strands resulting in a different blinking frequency and/or duration of the imaging nucleic acid strand when bound to the two different docking nucleic acid strands. For example, after cleaving, the imaging nucleic acid strand may exhibit stronger binding to docking nucleic acid strand 1 than docking nucleic acid strand 2, such that the target molecules associated with docking nucleic acid strand 1 and docking nucleic acid strand 2 are distinguishable based on the difference in blinking frequency and/or duration of the imaging nucleic acid strand.

### Transient Complex

In some embodiments, the hybridizing results in a transient complex being formed between the imaging nucleic acid strand and the docking nucleic acid strand. In some embodiments, the transient complex has a lifetime of less than 1 second. In some embodiments, the imaging nucleic acid strand exhibits blinking behavior due to the transient complex formed between the imaging nucleic acid strand and the docking nucleic acid strand.

In some embodiments, the cleaving modulates binding properties of the transient complex, resulting in a change in frequency and/or duration of blinking of the imaging nucleic acid strand. In some embodiments, the cleaving modulates binding properties of the transient complex, causing the imaging nucleic acid strand to bind to a docking nucleic acid strand of another target molecule. In some embodiments, the cleaving disrupts a hairpin structure in the imaging nucleic acid strand and exposes a nucleotide sequence in the imaging nucleic acid strand, which causes the imaging nucleic acid strand to bind to a docking nucleic acid strand of another target molecule. In some embodiments, the cleaving modulates binding properties of the transient complex, causing the imaging nucleic acid strand to bind to a docking nucleic acid strand of another target molecule. In some embodiments, the kinetics of the blinking behavior is correlated with a concentration of the imaging nucleic acid strand.

### Target Molecule

In some embodiments, the target molecule comprises a cancer protein. In some embodiments, the target molecule comprises a cancer protein or a cancer target molecule. In some embodiments, the cancer protein or the cancer target molecule is involved in cancer or a cancer pathway. Non-limiting examples of cancer proteins and cancer target molecules are listed in Table 1. In some embodiments, the cancer protein or the cancer target molecule comprises a tumor antigen.

TABLE 1
Cancer Proteins and Cancer Target Molecules

Cadherin 6 (CDH6)
Neural cell adhesion molecule (NCAM) (NCAM1) (CD56)
4-1BB ligand (4-1BBL) (TNFSF9) (CD137L)
Activated leukocyte cell adhesion molecule (ALCAM) (CD166)
Activin receptor-like kinase 1 (ACVRL1) (ALK1) (HHT2)
ADAM metallopeptidase domain 9 (ADAM9)
Alpha fetoprotein (AFP)
Angiopoietin 2 (ANG2) (ANGPT2)
Angiotensin II type 1 (AT1) receptor (AGTR1)
Anti-Mullerian hormone receptor type II (AMHR2)
AXL receptor tyrosine kinase (AXL) (UFO)
B and T lymphocyte attenuator (BTLA)
B7-H3 (CD276)
BLyS (BAFF) receptors
Bone morphogenetic protein receptors
Butyrophilin subfamily 2 member A2 (BTN2A2); Butyrophilin subfamily
2 member A1 (BTN2A1)
Butyrophilin like 2 (BTNL2)
Butyrophilin subfamily 3 member Al (BTN3A1)
CA 125
CA19.9 (sialyl Lewis A)
Cadherin 17 (CDH17); Tumor necrosis factor (TNF) receptor superfamily
member 10b (TNFRSF10B) (DR5) (TRAILR2) (CD262)
Calreticulin (CALR)
Cancer/testis antigen 1B (NY-ESO-1) (CTAG1B)
Carbonic anhydrase IX (CAIX)
Carcinoembryonic antigen (CEA)-related cell adhesion molecule 1
(CEACAM1) (CD66a)
Carcinoembryonic antigen (CEA)-related cell adhesion molecule 5
(CEACAM5) (CD66e)
Carcinoembryonic antigen (CEA)-related cell adhesion molecule 6
(CEACAM6) (NCA) (CD66c)
CC chemokine receptor 2 (CCR2) (CD192); CC chemokine receptor 5
(CCR5) (CD195)
CC chemokine receptor 4 (CCR4) (CD194)
CC chemokine receptor 5 (CCR5) (CD195)
CC chemokine receptor 8 (CCR8)
CD160
CD19
CD20
CD200
CD22 (SIGLEC2)
CD24
CD248 endosialin (TEM1)
CD27
CD28
CD3
Carcinoembryonic antigen (CEA)
Silver homolog (SILV) (PMEL17) (GP100) (PMEL)
Epidermal growth factor receptor (EGFR) (ErbB1) (HER1)
Epidermal growth factor receptor 2 (HER2) (EGFR2) (ErbB2) (neu)
Epidermal growth factor receptor variant III (EGFRvIII)
FMS-like tyrosine kinase 3 (FLT3) (CD135)
G protein-coupled receptor family C group 5 member D (GPRC5D)
Guanylate cyclase C (GCC) (GUCY2C)
Interleukin-3 (IL-3) receptor alpha (CD123)
Melanoma-associated antigen A4 (MAGEA4)
Mucin 17, cell surface associated (MUC17) (MUC3)
Programmed cell death 1 ligand 1 (PD-L1) (B7-H1) (CD274)
Six-transmembrane epithelial antigen of prostate 1 (STEAP1)
Somatostatin receptor 2 (SSTR2)
Tumor necrosis factor (TNF) receptor superfamily member 17 (BCMA)
(TNFRSF17) (CD269)
CD30
Fc gamma receptor IIIa (CD16a) (FCGR3A) (FcgammaRIIIa)
CD33 (SIGLEC3)
CD37
CD38
CD3d molecule delta (CD3-TCR complex) (CD3D)
Survivin (BIRC5)
CD4
CD40
CD46
CD47
Mesothelin (MSLN)
CD5
CD52
CD55SC-1
CD70 (CD27L)
CD74 molecule major histocompatibility complex class II invariant
chain
CD79b molecule immunoglobulin-associated beta (CD79B) (B29)
CD9
CD96 molecule (CD96)
CDKN1A interacting zinc finger protein 1 (CIZ1)
Claudin 1 (CLDN1)
Claudin 18 (CLDN18)
Claudin 3 (CLDN3)
Claudin 6 (CLDN6)
CLDN18.2
Clusterin (CLU) (APOJ)
c-Mer proto-oncogene tyrosine kinase (MERTK)
TYRO3 protein tyrosine kinase (TYRO3) (Sky)
c-Met receptor tyrosine kinase (c-MET) (MET) (HGFR)
c-Met receptor tyrosine kinase (c-MET) (MET) (HGFR)
Collagen triple helix repeat containing 1 (CTHRC1)
Colony-stimulating factor 1 receptor (CSF1R) (C-FMS) (CD115)
Complement 5 (C5)
Complement 5a (C5a)
Complement factor H (factor H) (CFH)
Complement receptor 5A (C5AR1) (C5AR) (CD88)
Connective tissue growth factor (CTGF)
C-type lectin domain family 12 member A (CLEC12A) (CLL1) (CD371)
C-type lectin domain family 2 member D (CLEC2D) (LLT1)
CXC chemokine receptor 4 (CXCR4) (NPY3R)
CXC chemokine receptor 5 (CXCR5)
Cytotoxic T-lymphocyte associated protein 4 (CTLA-4) (CTLA4)
(CD152)
Programmed cell death 1 (PD-1) (PDCD1) (CD279)
Cytotoxic T-lymphocyte associated protein 4 (CTLA-4) (CTLA4)
(CD152)
Tumor necrosis factor (TNF) receptor superfamily member 4 (OX40)
(TNFRSF4) (CD134)
Delta like canonical Notch ligand 3 (DLL3)
Delta like canonical Notch ligand 4 (DLL4)
Delta like non-canonical Notch ligand 1 (DLK1)
Dickkopf homolog 1 (DKK1)
Dickkopf homolog 2 (DKK2)
Dickkopf homolog 3 (DKK3)
Dipeptidyl peptidase-4 (DPP-4) (CD26)
Ecto-5′-nucleotidase (CD73) (NT5E)
Ectonucleoside triphosphate diphosphohydrolase 1 (CD39) (ENTPD1)
EPH receptor A2 (EPHA2)
EPH receptor A3 (EPHA3)
Epidermal growth factor (EGF)
Epidermal growth factor receptor (EGFR) (ErbB1) (HER1)
Erb-b2 receptor tyrosine kinase 3 (HER3) (ERBB3) (EGFR3)
Epidermal growth factor receptor (EGFR) (ErbB1) (HER1)
Fc gamma receptor III (CD16) (FCGR3)
Leucine-rich repeat-containing G protein-coupled receptor 5 (LGR5)
(GPR49)
Mucin 1 (MUC1) (CD227)
Epidermal growth factor receptor 2 (HER2) (EGFR2) (ErbB2) (neu)
Angiopoietin 2 (ANG2) (ANGPT2)
Epidermal growth factor receptor variant III (EGFRvIII)
Epithelial cell adhesion molecule (EpCAM)
Epithelial membrane protein 2 (EMP2)
Erb-b2 receptor tyrosine kinase 3 (HER3) (ERBB3) (EGFR3)
Estrogen receptor alpha 36
Factor X (FX); Factor IX
Fc gamma receptor (FCGR)
Signal regulatory protein alpha (SIRPA) (CD172a) (SHPS-1)
Fc gamma receptor IIb (FCGR2B) (CD32B)
Fc receptor-like 5 (FCRL5) (FCRH5)
Fibroblast activation protein (FAP)
Fibroblast growth factor (FGF) receptor 2 (FGFR2) (KGFR) (CD332)
Fibroblast growth factor (FGF) receptor 2 b isoform (FGFR2b)
Fibroblast growth factor 23 (FGF23)
Fibronectin extra ED-B domain isoform (FnEDB) (oncofetal fibronectin)
FMS-like tyrosine kinase 3 (FLT3) (CD135)
Folate receptor 1 (FOLR1) (FR-alpha) (FOLR)
Frizzled homolog 10 (FZD10)
Frizzled homolog 7 (FZD7)
Fucosylated GM1 ganglioside
Galectin-9 (LGALS9)
Ganglioside (GD3)
Ganglioside GD2 (GD2)
GDNF family receptor alpha like (GFRAL)
Globohexaosylceramide (globo-H)
Glucocorticoid-induced tumor necrosis factor receptor (TNFR)-related
protein (GITR) (TNFRSF18)
Glypican 1 (GPC1)
Glypican 3 (GPC3)
GnRH/LHRH receptor (GNRHR)
Granulocyte macrophage colony-stimulating factor (GM-CSF) (CSF2)
Growth differentiation factor 15 (GDF15)
Heat shock protein 70 (Hsp70)
Heparin-binding EGF-like growth factor (HBEGF) (DTR)
Hepatocyte growth factor/scatter factor (HGF/SF)
Hyaluronidase 2 (HYAL2)
Immunoglobulin G1 (IgG1)
Immunoglobulin superfamily member 11 (IGSF11)
Immunoglobulin-like domain containing receptor 2 (ILDR2)
Inducible T cell co-stimulator (ICOS)
Programmed cell death 1 (PD-1) (PDCD1) (CD279)
Insulin-like growth factor-1 (IGF-1) receptor (IGF1R) (CD221)
Insulin-like growth factor-2 (IGF-2)
Integrin alpha(V) (CD51)
Integrin alpha(V)beta(6)
Integrin alpha(V)beta(8); Transforming growth factor (TGF) beta 1
(TGFB1)
Integrin beta(1) (CD29)
Integrin beta(6)
Interferon beta (IFNB1)
Interleukin-1 (IL-1) alpha
Interleukin-1 (IL-1) beta
Interleukin-1 (IL-1) receptor accessory protein (IL-1RAP)
Interleukin-11 (IL-11) receptor
Interleukin-12 (IL-12)
Interleukin-15 (IL-15) receptor alpha chain (IL-15RA)
Interleukin-2 (IL-2) receptor
Interleukin-2 (IL-2) receptor alpha chain (CD25) (IL2RA)
Interleukin-2 (IL-2) receptor gamma chain (IL2RG) (CD132)
Interleukin-2 (IL-2) receptor beta chain (IL2RB) (CD122)
Interleukin-2 (IL-2) receptor
Interleukin-3 (IL-3) receptor alpha (CD123)
Interleukin-6 (IL-6)
Interleukin-6 (IL-6) receptor (CD126)
Interleukin-7 (IL-7) receptor (CD127)
Interleukin-8 (IL-8) (CXCL8)
Kappa myeloma antigen (KMA)
Kidney associated antigen 1 (KAAG1)
Killer cell immunoglobulin-like receptor three domains long
cytoplasmic tail 2 (KIR3DL2) (CD158K)
Killer cell immunoglobulin-like receptor two domains long cytoplasmic
tail 1 (KIR2DL1)
Killer cell lectin like receptor G1 (KLRG1)
Killer cell lectin-like receptor subfamily C member 1 (NKG2A)
(CD159a) (KLRC1)
Kita-kyushu lung cancer antigen 1 (KK-LC-1) (CT83)
KRAS proto-oncogene, GTPase (KRAS) (K-Ras)
Leucine-rich repeat containing 15 (LRRC15)
Leucine-rich repeat containing 32 (LRRC32) (GARP)
Leucine-rich repeat-containing G protein-coupled receptor 5 (LGR5)
(GPR49)
Leukemia inhibitory factor (LIF)
Leukocyte immunoglobin-like receptor subfamily B member 4 (LILRB4)
(CD85k) (ILT3)
Leukocyte immunoglobulin-like receptor subfamily B member 2
(LILRB2) (LIR2)
Low-density lipoprotein receptor-related protein 5 (LRP5)
Low-density lipoprotein receptor-related protein 6 (LRP6)
Lymphocyte antigen 6 complex locus E (Ly6E)
Lymphocyte-activation gene 3 (LAG3) (CD223)
Lysine-specific demethylase 1 (LSD1) (KDM1A)
Lysosomal-associated membrane protein 1 (LAMP1) (CD107a)
Macrophage stimulating 1 receptor c-Met-related tyrosine kinase
(MST1R) (RON) (CD136) (CDw136)
Major histocompatibility complex class I G (HLA-G)
Major histocompatibility complex class II DR (HLA-DR)
Mannose receptor C type 2 (MRC2) (UPARAP) (ENDO180)
Matrix metalloproteinase 9 (MMP9)
Melanoma cell adhesion molecule (MCAM) (MUC18) (CD146)
Melanoma-associated antigen A4 (MAGEA4)
Melanoma-associated chondroitin sulfate proteoglycan (MCSP) (CSPG4)
(HMW-MAA)
Mesothelin (MSLN)
MHC class I polypeptide-related sequence A (MICA)
MHC class I polypeptide-related sequence B (MICB)
Motile sperm domain containing 2 (MOSPD2)
Mucin 1 (MUC1) (CD227)
Mucin 16 (MUC16) (CA125)
Mucin 5AC oligomeric mucus/gel-forming (MUC5AC)
Natural cytotoxicity triggering receptor 1 (NCR1) (NKP46) (CD335)
Sialic acid binding Ig like lectin 9 (SIGLEC9)
Nectin-4 (PRR4)
Interleukin-2 (IL-2) receptor beta chain (IL2RB) (CD122)
Neonatal Fc receptor (FCRN) (FCGRT)
Netrin 1 (NTN1)
Neuropilin 1 (NRP1)
Neutrophil elastase (ELANE) (NE) (HLE)
Nicotinamide phosphoribosyltransferase (NamPRT) (NAMPT)
NLR family pyrin domain containing 3 (NLRP3) (NALP3) (CIAS1)
Non-functioning P2X7 receptor (nfP2X7)
Nucleolin
Oncofetal antigen 5T4
OX40 ligand (OX40L) (CD134L)
P selectin glycoprotein ligand-1 (PSGL-1) (CD162) (SELPLG)
Pancreatic adenocarcinoma upregulated factor (PAUF) (ZG16B)
P-cadherin (CDH3)
Periostin (POSTN)
Phosphatidylserine
Phosphodiesterase-I beta (ENPP3) (NPP3) (CD203c)
Phosphoinositide 3-kinase (PI3K)
Placental growth factor (PGF) (PLGF)
Plasminogen activator urokinase receptor (PLAUR) (uPAR) (suPAR)
Platelet derived growth factor receptor A (PDGFRA) (PDGFR2)
(CD140A)
Poliovirus receptor related immunoglobulin domain containing (PVRIG)
(CD112R)
Polymorphic epithelial mucin (PEM) (MUC-1)
Pre-B lymphocyte gene 1 (VPREB1)
Adenosine A2A receptor (ADORA2A) (A2AR) (ADORA2)
CD47
Cytotoxic T-lymphocyte associated protein 4 (CTLA-4) (CTLA4)
(CD152)
Lymphocyte-activation gene 3 (LAG3) (CD223)
Vascular endothelial growth factor (VEGF) receptor 1 (FLT1)
(VEGFR-1); Vascular endothelial growth factor (VEGF) receptor 3
(FLT4) (VEGFR-3)
Interleukin-2 (IL-2) receptor beta chain (IL2RB) (CD122)
Transforming growth factor (TGF) beta 1 (TGFB1)
Tumor necrosis factor (TNF) receptor superfamily member 4 (OX40)
(TNFRSF4) (CD134)
T cell immunoglobulin and mucin domain 3 (TIM3) (HAVCR2)
Prolactin receptor (PRLR)
Prostate-specific membrane antigen (PSMA) (FOLH1) (GCPII)
Protein tyrosine kinase 7 (PTK7) (CCK4)
Protein tyrosine phosphatase receptor type C (PTPRC) (CD45) (CD45R)
Receptor activator of NF-kappa B ligand (RANKL) (TNFSF11)
Receptor tyrosine kinase-like orphan receptor 1 (ROR1)
Receptor tyrosine kinase-like orphan receptor 2 (ROR2)
R-Spondin 3 (RSPO3)
S100 calcium binding protein A4 (S100A4)
S100 calcium binding protein A7 (S100A7) (psoriasin)
S100 calcium binding protein P (S100P)
Secretogranin III (SCG3)
Semaphorin 4D (SEMA4D)
Sialic acid binding Ig like lectin 15 (SIGLEC15)
Sialophorin (CD43) (SPN)
Signal regulatory protein alpha (SIRPA) (CD172a) (SHPS-1)
SLAM family member 7 (SLAMF7) (CS1)
Solute carrier family 3 member 2 (SLC3A2) (CD98)
Solute carrier family 34 sodium phosphate member 2 (SLC34A2)
(NaPi2b)
Solute carrier family 39 zinc transporter member 6 (SLC39A6) (LIV-1)
(ZIP6)
Solute carrier family 7 member 5 (SLC7A5) (LAT1)
Somatostatin receptor
Stabilin 1 (STAB1) (CLEVER-1) (FEEL-1)
Stage-specific embryonic antigen-4 (SSEA-4)
Stem cell factor (SCF) receptor tyrosine kinase (c-Kit) (KIT) (CD117)
Surface antigen in leukemia (SAIL)
T cell immunoglobulin and mucin domain 3 (TIM3) (HAVCR2)
T cell immunoreceptor with Ig and ITIM domains (TIGIT)
Tenascin C (TNC) (TN)
Thomsen-Friedenreich antigen (TF)
Tissue factor (TF) (CD142) (thromboplastin)
Tn antigen
Toll-like receptor 7 (TLR7)
Toll-like receptor 8 (TLR8)
Topoisomerase I (TOP1)
Tumor-associated calcium signal transducer 2 (TROP2) (TACSTD2)
(EGP-1)
Transferrin receptor (TFRC) (TFR) (CD71)
Transforming growth factor (TGF) beta 1 (TGFB1)
Transforming growth factor (TGF) beta 1 (TGFB1)
Transforming growth factor (TGF) beta 2 (TGFB2)
Transforming growth factor (TGF) beta 3 (TGFB3)
Triggering receptor expressed on myeloid cells 1 (TREM1)
Triggering receptor expressed on myeloid cells 2 (TREM2)
Tumor necrosis factor (TNF) receptor superfamily member 10a
(TNFRSF10A) (DR4) (TRAILR1) (CD261)
Tumor necrosis factor (TNF) receptor superfamily member 10b
(TNFRSF10B) (DR5) (TRAILR2) (CD262)
Tumor necrosis factor (TNF) receptor superfamily member 17 (BCMA)
(TNFRSF17) (CD269)
Fc gamma receptor IIIa (CD16a) (FCGR3A) (FcgammaRIIIa)
Tumor necrosis factor (TNF) receptor superfamily member 25
(TNFRSF25) (DR3) (LARD) (TRAMP) (WSL-1)
Tumor necrosis factor (TNF) receptor superfamily member 4 (OX40)
(TNFRSF4) (CD134)
Tumor necrosis factor (TNF) receptor superfamily member 9 (4-1BB)
(TNFRSF9) (CD137)
Tumor necrosis factor receptor 1 (TNFR1) (TNFRSF1A) (CD120a)
Tumor necrosis factor receptor 2 (TNFR2) (TNFRSF1B)
Tumor necrosis factor receptor superfamily member 13b (TNFRSF13B)
(CD267) (TACI)
Tumor necrosis factor (TNF) receptor superfamily member 17 (BCMA)
(TNFRSF17) (CD269)
Tumor necrosis factor receptor superfamily member 13c (BAFF-R)
(TNFRSF13C)
Tumor-associated antigen GBU4-5 (GBU4-5)
Tumor-associated calcium signal transducer 2 (TROP2) (TACSTD2)
(EGP-1)
Tumor-associated gp240
Vascular endothelial growth factor (VEGF)
Vascular endothelial growth factor (VEGF) receptor 2 (VEGFR-2)
(KDR/Flk-1)
Tyrosine kinase receptor 2 (Tie2)
Placental growth factor (PGF) (PLGF)
Vascular endothelial growth factor A (VEGF-A)
Neuropilin 1 (NRP1)
Vimentin (VIM)
V-region immunoglobulin-containing suppressor of T cell activation
(VISTA)
V-set domain containing T cell activation inhibitor 1 (B7-H4) (VTCN1)
Wilms tumor 1 (WT1)

In some embodiments, the target molecule comprises a target antigen.

In some embodiments, the target molecule comprises a target antigen.

In some embodiments, the target molecule comprises a ligand. In some embodiments, the ligand comprises a small molecule, a polymer, or a protein. In some embodiments, the target molecule comprises a lipid. In some embodiments, the target molecule comprises a carbohydrate.

### Labeling

In some embodiments, labeling the target molecule comprises directly connecting the docking nucleic acid strand to the target molecule. In some embodiments, labeling the target molecule comprises connecting the docking nucleic acid strand to the target molecule via a linker. In some embodiments, labeling the target molecule comprises binding a binding molecule to the target molecule, wherein the binding molecule selectively binds to the target molecule, and wherein the binding molecule is connected to the docking nucleic acid strand. In some embodiments, labeling the target molecule comprises connecting the docking nucleic acid strand to the binding molecule by a linker.

### Linker

In some embodiments, the linker connecting the binding molecule to the docking nucleic acid strand comprises a polypeptide. In some embodiments, the linker comprises a flexible linker. In some embodiments, the linker comprises a rigid linker. In some embodiments, the linker comprises a polymer. In some embodiments, the linker connects the N-terminus or the C-terminus of the binding molecule with the 5′-end or the 3′-end of the docking nucleic acid strand. In some embodiments, the linker connects the N-terminus of the binding molecule to the 5′-end of the docking nucleic acid strand. In some embodiments, the linker connects the N-terminus of the binding molecule to the 3′-end of the docking nucleic acid strand. In some embodiments, the linker connects the C-terminus of the binding molecule to the 5′-end of the docking nucleic acid strand. In some embodiments, the linker connects the C-terminus of the binding molecule to the 3′-end of the docking nucleic acid strand.

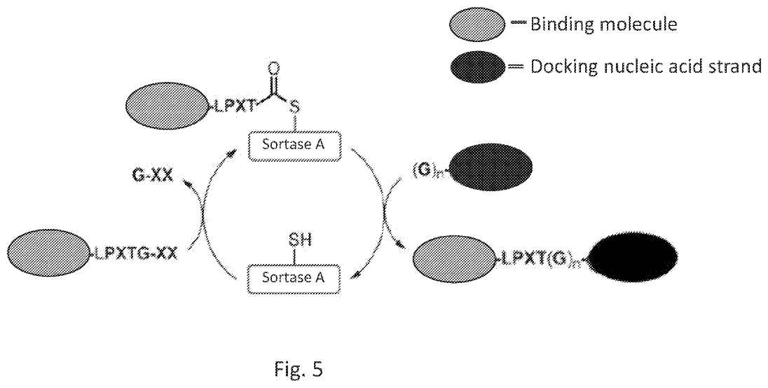

In some embodiments, the binding molecule and the docking nucleic acid strand are connected via a sortase-mediated ligation (see FIG. 5). In some embodiments, the sortase-mediated ligation comprises ligating a C-terminal LPXTG-XX sequence of the binding molecule with a GGG peptide at the 5′-end of the docking nucleic acid strand, wherein X is any amino acid (see FIG. 5). In some embodiments, the binding molecule is connected to the docking nucleic acid strand via a side chain of the binding molecule. In some embodiments, the side chain is an arginine side chain, a lysine side chain, or a cysteine side chain.

### Binding Molecule

In some embodiments, the binding molecule selectively binds the target molecule. In some embodiments, the binding molecule is selected from an antibody, a vhh antibody, a single-chain variable fragment (scFv), a Fab antibody fragment, a Fab antibody, a secondary antibody, a ligand, and a cofactor. In some embodiments, the binding molecule comprises an antibody. In some embodiments the binding molecule comprises a vhh antibody. In some embodiments, the binding molecule comprises a scFv. In some embodiments, the binding molecule comprises a Fab antibody fragment. In some embodiments, the binding molecule comprises a Fab antibody. In some embodiments, the binding molecule comprises a secondary antibody.

In some embodiments, the binding molecule comprises a ligand. In some embodiments, the ligand comprises a small molecule or a protein.

### Composition

In an aspect, the present disclosure provides cell samples for super-resolution microscopy (SRM). In some embodiments, a cell sample for SRM imaging comprises a target molecule expressed on cell surfaces of the cell sample; a docking nucleic acid strand associated with the target molecule; and an imaging nucleic acid strand hybridized to the docking nucleic acid strand, wherein the imaging nucleic acid strand includes a fluorophore and a cleavable moiety.

In some embodiments, the cleavable moiety is cleavable with light. In some embodiments, the cleavable moiety is cleavable with UV light. In some embodiments, the cleavable moiety has the structure shown in FIG. 3. In some embodiments, the cleavable moiety is cleavable with UV light having a wavelength in a range of about 300 nanometers (nm) to about 350 nm. In some embodiments, cleaving the cleavable moiety with light or UV light does not damage the cell sample.

In some embodiments, the cleavable moiety is cleavable by chemical cleavage. In some embodiments, the cleavable moiety is cleavable by enzymatic cleavage. In some embodiments, the cleavable moiety is cleavable with a nuclease.

In some embodiments, the cleavable moiety is located between the fluorophore and the imaging nucleic acid strand. In some embodiments, the cleavable moiety is located at or near the 5′-end of the imaging nucleic acid strand. In some embodiments, the cleavable moiety is located at or near the 3′-end of the imaging nucleic acid strand. In some embodiments, the cleavable moiety is located between the 5′-end and the 3′-end of the imaging nucleic acid strand. In some embodiments, the cleavable moiety is located within the sequence of the imaging nucleic acid strand. In some embodiments, the cleavable moiety is located between nucleotides of the imaging nucleic acid strand.

In some embodiments, the imaging nucleic acid strand includes at least two cleavable moieties. In some embodiments, the at least two cleavable moieties are independently cleavable.

In some embodiments, the fluorophore is located at the 5′-end of the imaging nucleic acid strand. In some embodiments, the fluorophore is located at the 3′-end of the imaging nucleic acid strand. In some embodiments, the fluorophore is located between the 5′-end and the 3′-end of the imaging nucleic acid strand. In some embodiments, the fluorophore is located at or near a middle of the imaging nucleic acid strand.

Non-limiting examples of a fluorophore includes, but are not limited to, abberior STAR RED, abberior STAR 635P, abberior STAR 635, ATTO 647N, ATTO 643, ATTO 633, abberior STAR ORANGE, abberior STAR 580, abberior STAR 600, ATTO 594, ATTO 590, abberior STAR GREEN, Oregon Green 488, abberior STAR 488, ATTO 488, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5-(or 6-) iodoacetamidofluorescein, 5-{[2 (and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, quantum dots, or other fluorophores.

In some embodiments, the imaging nucleic acid strand and the docking nucleic acid strand are single-stranded nucleic acid strands. In some embodiments, the imaging nucleic acid strand and the docking nucleic acid strand are complementary to each other. In some embodiments, the imaging nucleic acid strand comprises deoxyribonucleic acid (DNA). In some embodiments, the imaging nucleic acid strand comprises peptide nucleic acid (PNA). In some embodiments, the docking nucleic acid strand comprises DNA. In some embodiments, the docking nucleic acid strand comprises PNA. In some embodiments, one or both of the imaging nucleic acid strand and the docking nucleic acid strand comprise PNA. In some embodiments, one or both of the docking nucleic acid strand and the imaging nucleic acid strand comprise ribonucleic acid (RNA). In some embodiments, one or both of the docking nucleic acid strand and the imaging nucleic acid strand comprise an aptamer.

In some embodiments, one or both of the docking nucleic acid strand and the imaging nucleic acid strand comprise one or more non-natural nucleic acids. In some embodiments, the one or more non-natural nucleic acids comprise a non-natural nucleic acid described in Appella (Appella, D. H., “Non-natural Nucleic Acids for Synthetic Biology”, Curr. Opin. Chem. Biol. 2009, 13 (5-6), 687-696). In some embodiments, the one or more non-natural nucleic acids comprise one or more phosphorothionate linkages. In some embodiments, the one or more non-natural nucleic acids comprise one or more boranophosphate linkages. In some embodiments, the one or more non-natural nucleic acids comprise one or more phosphonate linkages. In some embodiments, the one or more non-natural nucleic acids comprise threose nucleic acid (TNA). In some embodiments, the one or more non-natural nucleic acids comprise locked nucleic acid (LNA). In some embodiments, the one or more non-natural nucleic acids comprise hexitol nucleic acid (HNA). In some embodiments, the one or more non-natural nucleic acids comprise flexible nucleic acid (FNA). In some embodiments, the one or more non-natural nucleic acids comprise glycerol nucleic acid (GNA). In some embodiments, the one or more non-natural nucleic acids comprise a nucleobase analog.

In some embodiments, the cell sample comprises a tissue sample. In some embodiments, the tissue sample is fixed. In some embodiments, the tissue sample is a live tissue sample. In some embodiments, the cell sample comprises cultured cells. In some embodiments, the cultured cells are fixed. In some embodiments, the cultured cells are live. In some embodiments, the cell sample comprises disseminated cells. In some embodiments, the cell sample comprises tissue fragments.

In some embodiments, the cell sample comprises cancer cells. In some embodiments, the cell sample comprises non-cancerous cells. In some embodiments, the cell sample comprises epithelial cells. In some embodiments, the cell sample comprises nerve cells. In some embodiments, the cell sample comprises muscle cells. In some embodiments, the cell sample comprises connective tissue cells. In some embodiments, the cell sample comprises normal (non-cancerous) or cancerous breast tissue cells. In some embodiments, the cell sample comprises a normal or cancerous lung tissue cells.

In some embodiments, the docking nucleic acid strand and the imaging nucleic acid strand form a stable complex. In some embodiments, the stable complex has a lifetime of at least about 1 second. In some embodiments, the stable complex has a lifetime of at least about 1 hour. In some embodiments, the stable complex has a lifetime of more than 1 hour. In some embodiments, the imaging nucleic acid strand exhibits non-blinking behavior due to the stable complex formed between the imaging nucleic acid strand and the docking nucleic acid strand. In some embodiments, the docking nucleic acid strand and the imaging nucleic acid strand form a transient complex. In some embodiments, the transient complex has a lifetime of less than about 1 second. In some embodiments, the imaging nucleic acid strand exhibits blinking behavior due to the transient complex formed between the imaging nucleic acid strand and the docking nucleic acid strand.

In some embodiments, the target molecule comprises a cancer protein. In some embodiments, the target molecule comprises a cancer protein or a cancer target molecule. In some embodiments, the cancer protein or the cancer target molecule is involved in cancer or a cancer pathway. Non-limiting examples of cancer proteins and cancer target molecules are listed in Table 1. In some embodiments, the cancer protein or the cancer target molecule comprises a tumor antigen. In some embodiments, the target molecule comprises a target antigen.

In some embodiments, the target molecule comprises a ligand. In some embodiments, the ligand comprises a small molecule or a protein. In some embodiments, the target molecule comprises a lipid. In some embodiments, the target molecule comprises a carbohydrate.

In some embodiments, the target molecule is directly connected to the docking nucleic acid strand. In some embodiments, the target molecule is connected to the docking nucleic acid strand by a linker.

In some embodiments, the docking nucleic acid strand is connected to a binding molecule by a linker, and wherein the binding molecule is selectively bound to the target molecule. In some embodiments, the binding molecule is selected from an antibody, a vhh antibody, a single-chain variable fragment (scFv), a Fab antibody fragment, a Fab antibody, a secondary antibody, a ligand, and a cofactor. In some embodiments, the binding molecule is an antibody. In some embodiments, the binding molecule is a vhh antibody. In some embodiments, the binding molecule is a scFv. In some embodiments, the binding molecule is a Fab antibody fragment. In some embodiments, the binding molecule is a Fab antibody. In some embodiments, the binding molecule is a ligand. In some embodiments, the ligand comprises a small molecule or a protein.

In some embodiments, the linker connecting the docking nucleic acid strand to the binding molecule comprises a polypeptide. In some embodiments, the linker is a flexible linker. In some embodiments, the linker is a rigid linker. In some embodiments, the binding molecule is connected to the docking nucleic acid strand via a side chain of the binding molecule. In some embodiments, the side chain is an arginine side chain, a lysine side chain, or a cysteine side chain.

### EMBODIMENTS LISTING

Embodiment 1 comprises a method of providing a super-resolution microscopy (SRM) image of a target molecule expressed on cell surfaces of a cell sample, comprising: a) labeling the target molecule with a docking nucleic acid strand; b) hybridizing an imaging nucleic acid strand to the docking nucleic acid strand, wherein the imaging nucleic acid strand includes a fluorophore and at least one cleavable moiety, and wherein the hybridizing brings the fluorophore in proximity to the target molecule; c) imaging the cell sample to provide the SRM image of the target molecule; and d) cleaving the cleavable moiety to displace the fluorophore from proximity to the target molecule, to change a blinking behavior of the imaging nucleic acid strand, and/or to change a binding specificity of the imaging nucleic acid strand.
Embodiment 2 comprises the method of embodiment 1, wherein cleaving the cleavable moiety displaces the fluorophore from proximity to the target molecule.
Embodiment 3 comprises the method of embodiments 1 or 2, further comprising providing a multiplexed image of a number (n) of different target molecules expressed on the cell surfaces of the cell sample by repeating steps a) to d) for each of the n different target molecules to provide an SRM image of each of the n different target molecules; and combining the SRM images of the n different target molecules to provide the multiplexed image.
Embodiment 4 comprises a method of providing a multiplexed super-resolution microscopy (SRM) image of a number (n) of different target molecules expressed on cell surfaces of a cell sample comprising: i) labeling each of the different target molecules with a different docking nucleic acid strand; ii) obtaining a SRM image of at least one target molecule of the n different target molecules by: hybridizing an imaging nucleic acid strand to the docking nucleic acid strand associated with the target molecule, wherein the imaging nucleic acid strand includes a fluorophore and a cleavable moiety, and wherein the hybridizing brings the fluorophore of the imaging nucleic acid strand in proximity to the target molecule, imaging the cell sample using fluorescence emission of the fluorophore, and cleaving the cleavable moiety to remove the fluorophore from proximity to the target molecule, to change a blinking behavior of the imaging nucleic acid strand, and/or to change a binding specificity of the imaging nucleic acid strand; iii) repeating step ii) until SRM images of all of the n different target molecules of the cell sample are obtained; and iv) combining the SRM images of all of the n different target molecules to provide the multiplexed SRM image of the n different target molecules expressed on the cell surfaces of the cell sample.
Embodiment 5 comprises the method of any one of embodiments 1-4, wherein imaging the cell sample comprises imaging the cell sample by stimulated emission depletion (STED) microscopy, ground state depletion (GSD) microscopy, MINFLUX microscopy, stochastic optical reconstruction microscopy (STORM), direct stochastic optical reconstruction microscopy (DSTORM), or single-molecule localization microscopy.
Embodiment 6 comprises the method of any one of embodiments 3-5, wherein a different imaging nucleic acid strand is used for imaging each of the n different target molecules.
Embodiment 7 comprises the method of any one of embodiments 3-5, wherein one imaging nucleic acid strand is used to image a subset of two or more of the n different target molecules.
Embodiment 8 comprises the method of embodiments 4 or 5, wherein the step of ii) obtaining the SRM image comprises obtaining an SRM image of a subset of target molecules of the n different target molecules simultaneously.
Embodiment 9 comprises the method of embodiment 8, wherein a different imaging nucleic acid strand with a different fluorophore is used for imaging each of the target molecules in the subset.
Embodiment 10 comprises the method of embodiments 8 or 9, wherein the subset includes at least 2 different target molecules.
Embodiment 11 comprises the method of embodiments 8 or 9, wherein the subset includes at least 3 different target molecules.
Embodiment 12 comprises the method of any one of embodiments 3 to 11, wherein n is at least 10.
Embodiment 13 comprises the method of any one of embodiments 3 to 11, wherein n is at least 20.
Embodiment 14 comprises the method of any one of embodiments 3 to 11, wherein n is at least 50.
Embodiment 15 comprises the method of any one of embodiments 3 to 11, wherein n is at least 100.
Embodiment 16 comprises the method of any one of the preceding embodiments, wherein cleaving the cleavable moiety comprises cleaving the cleavable moiety with light.
Embodiment 17 comprises the method of any one of the preceding embodiments, wherein cleaving the cleavable moiety comprises cleaving the cleavable moiety with UV light.
Embodiment 18 comprises the method of any one of embodiments 1-15, wherein cleaving the cleavable moiety comprises cleaving the cleavable moiety via chemical cleavage.
Embodiment 19 comprises the method of any one of embodiments 1-15, wherein cleaving the cleavable moiety comprises cleaving the cleavable moiety via enzymatic cleavage.
Embodiment 20 comprises the method of any one of embodiments 1-15, wherein cleaving the cleavable moiety comprises cleaving the cleavable moiety with a nuclease.
Embodiment 21 comprises the method of any one of the preceding embodiments, wherein cleaving the cleavable moiety releases the fluorophore from the imaging nucleic acid strand.
Embodiment 22 comprises the method of any one of the preceding embodiments, wherein cleaving the cleavable moiety releases at least a portion of the imaging nucleic acid strand from the docking nucleic acid strand.
Embodiment 23 comprises the method of any one of the preceding embodiments, further comprising displacing the imaging nucleic acid strand from the docking nucleic acid strand by hybridizing a non-labeled nucleic acid strand to the docking nucleic acid strand after cleaving the cleavable moiety.
Embodiment 24 comprises the method of any one of embodiments 1-22, wherein cleaving the cleavable moiety releases the imaging nucleic acid strand from the docking nucleic acid strand.
Embodiment 25 comprises the method of any one of embodiments 1-23, wherein cleaving the cleavable moiety changes kinetics of dissociation of the imaging nucleic acid strand from the docking nucleic acid strand, thereby changing the blinking behavior of the imaging nucleic acid strand.
Embodiment 26 comprises the method of any one of the preceding embodiments, wherein cleaving the cleavable moiety changes the binding specificity of the imaging nucleic acid strand for the docking nucleic acid strand.
Embodiment 27 comprises the method of any one of the preceding embodiments, wherein the cleavable moiety is located between the fluorophore and the imaging nucleic acid strand, the cleavable moiety is located in or is part of the fluorophore, the cleavable moiety is in the docking nucleic acid strand, the cleavable moiety is between a binding molecule and the fluorophore wherein the binding molecule binds to the target molecule and is connected to the docking nucleic acid strand, or the cleavable moiety is in the imaging nucleic acid strand.
Embodiment 28 comprises the method of any one of embodiments 1-26, wherein the cleavable moiety is located within the sequence of the imaging nucleic acid strand.
Embodiment 29 comprises the method of any one of embodiments 1-26, wherein the cleavable moiety is located between nucleotides of the imaging nucleic acid strand.
Embodiment 30 comprises the method of any one of the preceding embodiments, wherein the imaging nucleic acid strand includes at least two cleavable moieties, and optionally wherein the at least two cleavable moieties are located in the imaging nucleic acid strand, in the docking nucleic acid strand, between the binding molecule and the fluorophore, or in the fluorophore.
Embodiment 31 comprises the method of any one of the preceding embodiments, wherein the imaging nucleic acid strand includes at least two cleavable moieties that are independently cleavable.
Embodiment 32 comprises the method of any one of the preceding embodiments, wherein the hybridizing brings the fluorophore within a distance of about 2 nanometers (nm) to about 10 nm from the target molecule.
Embodiment 33 comprises the method of any one of the preceding embodiments, wherein the fluorophore is located at the 5′-end of the imaging nucleic acid strand.
Embodiment 34 comprises the method of any one of embodiments 1-32, wherein the fluorophore is located at the 3′-end of the imaging nucleic acid strand.
Embodiment 35 comprises the method of any one of embodiments 1-32, wherein the fluorophore is located between the 5′-end and the 3′-end of the imaging nucleic acid strand.
Embodiment 36 comprises the method of any one of embodiments 1-32, wherein the fluorophore is located at or near a middle of the imaging nucleic acid strand.
Embodiment 37 comprises the method of any one of the preceding embodiments, wherein the imaging nucleic acid strand comprises deoxyribonucleic acid (DNA).
Embodiment 38 comprises the method of any one of the preceding embodiments, wherein the imaging nucleic acid strand comprises peptide nucleic acid (PNA).
Embodiment 39 comprises the method of any one of the preceding embodiments, wherein the docking nucleic acid strand comprises deoxyribonucleic acid (DNA).
Embodiment 40 comprises the method of any one of the preceding embodiments, wherein the docking nucleic acid strand comprises peptide nucleic acid (PNA).
Embodiment 41 comprises the method of any one of the preceding embodiments, wherein one or both of the docking nucleic acid strand and the imaging nucleic acid strand comprise ribonucleic acid (RNA).
Embodiment 42 comprises the method of any one of the preceding embodiments, wherein one or both of the docking nucleic acid strand and the imaging nucleic acid strand comprise an aptamer.
Embodiment 43 comprises the method of any one of the preceding embodiments, wherein one or both of the docking nucleic acid strand and the imaging nucleic acid strand comprise one or more non-natural nucleic acids.
Embodiment 44 comprises the method of embodiment 43, wherein the one or more non-natural nucleic acids comprise one or more phosphorothionate backbone linkages.
Embodiment 45 comprises the method of embodiment 43, wherein the one or more non-natural nucleic acids comprise one or more boranophosphate backbone linkages.
Embodiment 46 comprises the method of embodiment 43, wherein the one or more non-natural nucleic acids comprise one or more phosphonate backbone linkages.
Embodiment 47 comprises the method of embodiment 43, wherein the one or more non-natural nucleic acids comprise threose nucleic acid (TNA).
Embodiment 48 comprises the method of embodiment 43, wherein the one or more non-natural nucleic acids comprise locked nucleic acid (LNA).
Embodiment 49 comprises the method of embodiment 43, wherein the one or more non-natural nucleic acids comprise hexitol nucleic acid (HNA).
Embodiment 50 comprises the method of embodiment 43, wherein the one or more non-natural nucleic acids comprise flexible nucleic acid (FNA).
Embodiment 51 comprises the method of embodiment 43, wherein the one or more non-natural nucleic acids comprise glycerol nucleic acid (GNA).
Embodiment 52 comprises the method of embodiment 43, wherein the one or more non-natural nucleic acids comprise a nucleobase analog.
Embodiment 53 comprises the method of any one of the preceding embodiments, further comprising fixing the cell sample prior to labeling the target molecule.
Embodiment 54 comprises the method of any one of the preceding embodiments, wherein the cell sample comprises disseminated cells.
Embodiment 55 comprises the method of any one of the preceding embodiments, wherein the cell sample comprises tissue fragments.
Embodiment 56 comprises the method of any one of the preceding embodiments, wherein the cell sample comprises a tissue sample.
Embodiment 57 comprises the method of embodiment 56, wherein the tissue sample is fixed.
Embodiment 58 comprises the method of embodiment 56, wherein the tissue sample is a live tissue sample.
Embodiment 59 comprises the method of any one of embodiments 1-53, wherein the cell sample comprises cultured cells.
Embodiment 60 comprises the method of embodiment 59, wherein the cultured cells are fixed.
Embodiment 61 comprises the method of embodiment 59, wherein the cultured cells are live.
Embodiment 62 comprises the method of any one of the preceding embodiments, wherein the method is carried out without changing buffer conditions of the cell sample.
Embodiment 63 comprises the method of any one of the preceding embodiments, wherein all of the steps of the method are carried out in a same buffer, or wherein buffer conditions and/or concentration of the binding molecule and/or the imaging nucleic acid strand is changed at each step.
Embodiment 64 comprises the method of any one of the preceding embodiments, wherein the method is carried out in physiological buffer conditions.
Embodiment 65 comprises the method of any of the preceding embodiments, wherein all of the steps of the method are carried out in a single buffer having a physiological pH and a physiological salt concentration.
Embodiment 66 comprises the method of any one of the preceding embodiments, wherein all of the steps of the method are carried out in a single buffer having a pH in a range of about 7.2 to about 7.5, and a sodium chloride concentration in a range of about 135 millimolar (mM) to about 145 mM.
Embodiment 67 comprises the method of any one of the preceding embodiments, wherein the method is carried out in a buffer having a sodium chloride concentration in a range of about 135 millimolar (mM) to about 145 mM.
Embodiment 68 comprises the method of any one of the preceding embodiments, wherein all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4.
Embodiment 69 comprises the method of any one of embodiments 1-61, wherein one or more steps of the method are carried out in a buffer having a salt concentration of about 500 millimolar (mM).
Embodiment 70 comprises the method of any one of embodiments 1-61, wherein one or more of the steps of the method are carried out in a buffer having a salt concentration in a range of about 500 millimolar (mM) to about 600 mM.
Embodiment 71 comprises the method of any one of embodiments 1-61, wherein one or more steps of the method are carried out in a buffer having a salt concentration of about 50 millimolar (mM).
Embodiment 72 comprises the method of any one of the preceding embodiments, wherein the cleaving removes the fluorophore from the imaging nucleic acid strand.
Embodiment 73 comprises the method of embodiment 72, further comprising washing the cell sample after the cleaving, or diluting the docking nucleic acid strand or the imaging nucleic acid strand or the fluorophore with buffer after the cleaving.
Embodiment 74 comprises the method of embodiment 73, further comprising acquiring an SRM or confocal image of the cell sample after the washing to confirm removal of the fluorophore.
Embodiment 75 comprises the method of any one of the preceding embodiments, wherein the cleaving causes the imaging nucleic acid strand to dissociate from the docking nucleic acid strand.
Embodiment 76 comprises the method of any one of the preceding embodiments, wherein the cleaving causes the imaging nucleic acid strand to expose a different sequence of nucleotide that binds to a different docking nucleic acid strand.
Embodiment 77 comprises the method of any one of embodiments 1-71, wherein the cleaving removes the fluorophore and at least a portion of the imaging nucleic acid strand from the docking nucleic acid strand.
Embodiment 78 comprises the method of embodiment 77, further comprising washing the cell sample after the cleaving.
Embodiment 79 comprises the method of embodiment 78, further comprising acquiring an SRM or confocal image of the cell sample after the washing to confirm removal of the fluorophore and the imaging nucleic acid strand from the docking nucleic acid strand.
Embodiment 80 comprises the method of any one of the preceding embodiments, wherein the hybridizing results in a stable complex being formed between imaging nucleic acid strand and the docking nucleic acid strand.
Embodiment 81 comprises the method of embodiment 80, wherein the stable complex has a lifetime of at least about 1 second.
Embodiment 82 comprises the method of embodiment 80, wherein the stable complex has a lifetime of at least about 1 hour.
Embodiment 83 comprises the method of embodiment 80, wherein the stable complex has a lifetime of more than 1 hour.
Embodiment 84 comprises the method of any one of embodiments 80-83, wherein the imaging nucleic acid strand exhibits non-blinking behavior due to the stable complex formed between the imaging nucleic acid strand and the docking nucleic acid strand.
Embodiment 85 comprises the method of any one of embodiments 80-84, wherein the cleaving destabilizes the stable complex formed between the imaging nucleic acid strand and the docking nucleic acid strand, thereby causing the imaging nucleic acid strand and the docking nucleic acid strand to form a transient complex, and causing the imaging nucleic acid strand to exhibit blinking behavior.
Embodiment 86 comprises the method of any one of embodiments 80-85, wherein the cleaving causes the imaging nucleic acid strand to exhibit a blinking behavior.
Embodiment 87 comprises the method of any one of embodiments 1-79, wherein the hybridizing results in a transient complex being formed between the imaging nucleic acid strand and the docking nucleic acid strand.
Embodiment 88 comprises the method of embodiment 87, wherein the imaging nucleic acid strand exhibits blinking behavior due to the transient complex formed between the imaging nucleic acid strand and the docking nucleic acid strand.
Embodiment 89 comprises the method of embodiments 87 or 88, wherein the cleaving modulates binding properties of the transient complex, resulting in a change in frequency and/or duration of blinking of the imaging nucleic acid strand.
Embodiment 90 comprises the method of any one of embodiments 87-89, wherein the cleaving modulates binding properties of the transient complex, causing the imaging nucleic acid strand to bind to a docking nucleic acid strand of another target molecule.
Embodiment 91 comprises the method of any one of embodiments 87-90, wherein kinetics of the blinking behavior is correlated with a concentration of the imaging nucleic acid strand.
Embodiment 92 comprises the method of any one of the preceding embodiments, wherein the target molecule comprises a cancer protein.
Embodiment 93 comprises the method of any one of the preceding embodiments, wherein the target molecule comprises a tumor antigen.
Embodiment 94 comprises the method of any one of embodiments 1-91, wherein the target molecule comprises a ligand.
Embodiment 95 comprises the method of any one of embodiments 1-91, wherein the target molecule comprises a lipid.
Embodiment 96 comprises the method of any one of embodiments 1-91, wherein the target molecule comprises a carbohydrate.
Embodiment 97 comprises the method of any one of the preceding embodiments, wherein labeling the target molecule comprises binding a binding molecule to the target molecule, wherein the binding molecule selectively binds to the target molecule, and wherein the binding molecule is connected to the docking nucleic acid strand, optionally wherein the cleavable moiety is located between the binding molecule and the fluorophore, and optionally wherein a concentration of the binding molecule is changed at each step.
Embodiment 98 comprises the method of any one of embodiments 1-96, wherein labeling the target molecule comprises directly connecting the docking nucleic acid strand to the target molecule.
Embodiment 99 comprises the method of embodiment 97, wherein labeling the target molecule comprises connecting the docking nucleic acid strand to the binding molecule by a linker, optionally wherein the at least one cleavable moiety is located between the binding molecule and the docking nucleic acid strand or in the linker, optionally wherein the at least one cleavable moiety is located between the binding molecule and the imaging nucleic acid strand or in the linker, or optionally wherein at least two cleavable moieties are in the linker.
Embodiment 100 comprises the method of embodiment 97, wherein the binding molecule is selected from an antibody, a vhh antibody, a single-chain variable fragment (scFv), a Fab antibody fragment, a Fab antibody, a secondary antibody, a ligand, and a cofactor.
Embodiment 101 comprises the method of embodiment 100, wherein the ligand comprises a small molecule, a peptide, or a protein.
Embodiment 102 comprises the method of embodiment 100, wherein the binding molecule is a vhh antibody.
Embodiment 103 comprises the method of embodiment 99, wherein the linker comprises a polypeptide.
Embodiment 104 comprises the method of any one of embodiments 99 to 103, wherein the linker connects the N-terminus or C-terminus of the binding molecule with the 5′-end or the 3′-end of the docking nucleic acid strand.
Embodiment 105 comprises the method of any one of embodiments 99-104, wherein the binding molecule and the docking nucleic acid strand are connected via a sortase-mediated ligation.
Embodiment 106 comprises the method of embodiment 105, wherein the sortase-mediated ligation comprises ligating a C-terminal LPXTG-XX sequence of the binding molecule with a GGG peptide at the 5′-end of the docking nucleic acid strand.
Embodiment 107 comprises the method of embodiment 97, wherein the binding molecule is connected to the docking nucleic acid strand via a side chain of the binding molecule.
Embodiment 108 comprises the method of embodiment 107, wherein the side chain is an arginine side chain or a cysteine side chain.
Embodiment 109 comprises a cell sample for super-resolution microscopy (SRM) imaging, comprising: a target molecule expressed on cell surfaces of the cell sample; a docking nucleic acid strand associated with the target molecule; and an imaging nucleic acid strand hybridized to the docking nucleic acid strand, wherein the imaging nucleic acid strand includes a fluorophore and a cleavable moiety.
Embodiment 110 comprises the cell sample of embodiment 109, wherein the cleavable moiety is cleavable with light.
Embodiment 111 comprises the cell sample of embodiment 109, wherein the cleavable moiety is cleavable with UV light.
Embodiment 112 comprises the cell sample of embodiment 109, wherein the cleavable moiety is cleavable by chemical cleavage.
Embodiment 113 comprises the cell sample of embodiment 109, wherein the cleavable moiety is cleavable by enzymatic cleavage.
Embodiment 114 comprises the cell sample of embodiment 109, wherein the cleavable moiety is cleavable with a nuclease.
Embodiment 115 comprises the cell sample of any one of embodiments 109-114, wherein: the cleavable moiety is located between the fluorophore and the imaging nucleic acid strand, the cleavable moiety is located in or is part of the fluorophore, the cleavable moiety is in the docking nucleic acid strand, the cleavable moiety is between a binding molecule and the fluorophore wherein the binding molecule binds to the target molecule and is connected to the docking nucleic acid strand, or the cleavable moiety is in the imaging nucleic acid strand.
Embodiment 116 comprises the cell sample of any one of embodiments 109-114, wherein the cleavable moiety is located within the sequence of the imaging nucleic acid strand.
Embodiment 117 comprises the cell sample of any one of embodiments 109-114, wherein the cleavable moiety is located between nucleotides of the imaging nucleic acid strand.
Embodiment 118 comprises the cell sample of any one of clams 109-117, wherein the imaging nucleic acid strand includes at least two cleavable moieties, and optionally wherein the at least two cleavable moieties are located in the imaging nucleic acid strand, in the docking nucleic acid strand, between the binding molecule and the fluorophore, or in the fluorophore.
Embodiment 119 comprises the cell sample of any one of embodiments 109-118, wherein the at least two cleavable moieties are independently cleavable.
Embodiment 120 comprises the cell sample of any one of embodiments 109-119, wherein the fluorophore is located at the 5′-end of the imaging nucleic acid strand.
Embodiment 121 comprises the cell sample of any one of embodiments 109-119, wherein the fluorophore is located at the 3′-end of the imaging nucleic acid strand.
Embodiment 122 comprises the cell sample of any one of embodiments 109-119, wherein the fluorophore is located between the 5′-end and the 3′-end of the imaging nucleic acid strand.
Embodiment 123 comprises the cell sample of any one of embodiments 109-119, wherein the fluorophore is located at or near a middle of the imaging nucleic acid strand.
Embodiment 124 comprises the cell sample of any one of embodiments 109-123, wherein the imaging nucleic acid strand comprises deoxyribonucleic acid (DNA).
Embodiment 125 comprises the cell sample of any one of embodiments 109-124, wherein the imaging nucleic acid strand comprises peptide nucleic acid (PNA).
Embodiment 126 comprises the cell sample of any one of embodiments 109-125, wherein the docking nucleic acid strand comprises deoxyribonucleic acid (DNA).
Embodiment 127 comprises the cell sample of any one of embodiments 109-126, wherein the docking nucleic acid strand comprises peptide nucleic acid (PNA).
Embodiment 128 comprises the cell sample of any one of embodiments 109-127, wherein one or both of the docking nucleic acid strand and the imaging nucleic acid strand comprises ribonucleic acid (RNA).
Embodiment 129 comprises the cell sample of any one of embodiments 109-127, wherein one or both of the docking nucleic acid strand and the imaging nucleic acid strand comprise an aptamer.
Embodiment 130 comprises the cell sample of any one of embodiments 109-129, wherein one or both of the docking nucleic acid strand and the imaging nucleic acid strand comprise one or more non-natural nucleic acids.
Embodiment 131 comprises the cell sample of embodiment 130, wherein the one or more non-natural nucleic acids comprise one or more phosphorothionate linkages.
Embodiment 132 comprises the cell sample of embodiment 130, wherein the one or more non-natural nucleic acids comprise one or more boranophosphate linkages.
Embodiment 133 comprises the cell sample of embodiment 130, wherein the one or more non-natural nucleic acids comprise one or more phosphonate linkages.
Embodiment 134 comprises the cell sample of embodiment 130, wherein the one or more non-natural nucleic acids comprise threose nucleic acid (TNA).
Embodiment 135 comprises the cell sample of embodiment 130, wherein the one or more non-natural nucleic acids comprise locked nucleic acid (LNA).
Embodiment 136 comprises the cell sample of embodiment 130, wherein the one or more non-natural nucleic acids comprise hexitol nucleic acid (HNA).
Embodiment 137 comprises the cell sample of embodiment 130, wherein the one or more non-natural nucleic acids comprise flexible nucleic acid (FNA).
Embodiment 138 comprises the cell sample of embodiment 130, wherein the one or more non-natural nucleic acids comprise glycerol nucleic acid (GNA).
Embodiment 139 comprises the cell sample of embodiment 130, wherein the one or more non-natural nucleic acids comprise a nucleobase analog.
Embodiment 140 comprises the cell sample of any one of embodiments 109-139, wherein the cell sample comprises a tissue sample.
Embodiment 141 comprises the cell sample of embodiment 140, wherein the tissue sample is fixed.
Embodiment 142 comprises the cell sample of embodiment 140, wherein the tissue sample is a live tissue sample.
Embodiment 143 comprises the cell sample of any one of embodiments 109-139, wherein the cell sample comprises cultured cells.
Embodiment 144 comprises the cell sample of embodiment 143, wherein the cultured cells are fixed.
Embodiment 145 comprises the cell sample of embodiment 143, wherein the cultured cells are live.
Embodiment 146 comprises the cell sample of any one of embodiments 109-139, wherein the cell sample comprises disseminated cells.
Embodiment 147 comprises the cell sample of any one of embodiments 109-139, wherein the cell sample comprises tissue fragments.
Embodiment 148 comprises the cell sample of any one of embodiments 109-147, wherein the docking nucleic acid strand and the imaging nucleic acid strand form a stable complex.
Embodiment 149 comprises the cell sample of embodiment 148, wherein the stable complex has a lifetime of at least about 1 second.
Embodiment 150 comprises the cell sample of embodiment 148, wherein the stable complex has a lifetime of at least about 1 hour.
Embodiment 151 comprises the cell sample of embodiment 148, wherein the stable complex has a lifetime of more than 1 hour.
Embodiment 152 comprises the cell sample of any one of embodiments 109-151, wherein the target molecule comprises a cancer protein.
Embodiment 153 comprises the cell sample of any one of embodiments 109-152, wherein the target molecule comprises a tumor antigen.
Embodiment 154 comprises the cell sample of any one of embodiments 109-151, wherein the target molecule comprises a ligand.
Embodiment 155 comprises the cell sample of any one of embodiments 109-151, wherein the target molecule comprises a lipid.
Embodiment 156 comprises the cell sample of any one of embodiments 109-151, wherein the target molecule comprises a carbohydrate.
Embodiment 157 comprises the cell sample of any one of embodiments 109-156, wherein the target molecule is directly connected to the docking nucleic acid strand.
Embodiment 158 comprises the cell sample of any one of embodiments 109-156, wherein the docking nucleic acid strand is connected to a binding molecule by a linker, and wherein the binding molecule is selectively bound to the target molecule, optionally wherein the at least one cleavable moiety is located between the binding molecule and the docking nucleic acid strand or in the linker, optionally wherein the cleavable moiety is located between the binding molecule and the fluorophore, and optionally wherein at least two cleavable moieties are in the linker.
Embodiment 159 comprises the cell sample of 158, wherein the binding molecule is selected from an antibody, a vhh antibody, a single-chain variable fragment (scFv), a Fab antibody fragment, a Fab antibody, a secondary antibody, a ligand, and a cofactor.
Embodiment 160 comprises the cell sample of embodiment 159, wherein the ligand comprises a small molecule, a peptide, or a protein.
Embodiment 161 comprises the cell sample of embodiment 159, wherein the binding molecule is a vhh antibody.
Embodiment 162 comprises the cell sample of any one of embodiments 158-161, wherein the linker comprises a polypeptide.
Embodiment 163 comprises the cell sample of any one of embodiments 158-162, wherein the binding molecule is connected to the docking nucleic acid strand via a side chain of the binding molecule.
Embodiment 164 comprises the cell sample of embodiment 163, wherein the side chain is an arginine side chain or a cysteine side chain.
Embodiment 165 comprises a method of providing a super-resolution microscopy (SRM) image of a target molecule expressed on cell surfaces of a cell sample, comprising: a) labeling the target molecule with an imaging acid strand, wherein the imaging nucleic acid strand includes a fluorophore and at least one cleavable moiety, and wherein the labeling of the target molecule brings the fluorophore in proximity to the target molecule; b) imaging the cell sample to provide the SRM image of the target molecule; and c) cleaving the cleavable moiety.
Embodiment 166 comprises the method of embodiment 165, wherein cleaving the cleavable moiety displaces the fluorophore from proximity to the target molecule.
Embodiment 167 comprises the method of embodiments 165 or 166, further comprising providing a multiplexed image of a number (n) of different target molecules expressed on the cell surfaces of the cell sample in a single step or by: repeating steps a) to c) for each of the n different target molecules to provide an SRM image of each of the n different target molecules; and combining the SRM images of the n different target molecules to provide the multiplexed image.
Embodiment 168 comprises the method of any one of embodiments 165 to 167, wherein imaging the cell sample comprises imaging the cell sample by stimulated emission depletion (STED) microscopy, ground state depletion (GSD) microscopy, MINFLUX microscopy, stochastic optical reconstruction microscopy (STORM), direct stochastic optical reconstruction microscopy (DSTORM), or single-molecule localization microscopy.
Embodiment 169 comprises the method of embodiment 167, wherein n is at least 10, n is at least 20, n is at least 50, or n is at least 100.
Embodiment 170 comprises the method of any one of embodiments 165 to 169, wherein cleaving the cleavable moiety comprises cleaving the cleavable moiety with light.
Embodiment 171 comprises the method of any one of embodiments 165 to 170, wherein cleaving the cleavable moiety comprises cleaving the cleavable moiety with UV light.
Embodiment 172 comprises the method of any one of embodiments 165 to 169, wherein cleaving the cleavable moiety comprises cleaving the cleavable moiety via chemical cleavage.
Embodiment 173 comprises the method of any one of embodiments 165 to 169, wherein cleaving the cleavable moiety comprises cleaving the cleavable moiety via enzymatic cleavage.
Embodiment 174 comprises the method of any one of embodiments 165 to 169, wherein cleaving the cleavable moiety comprises cleaving the cleavable moiety with a nuclease.
Embodiment 175 comprises the method of any one of embodiments 165 to 174, wherein cleaving the cleavable moiety releases the fluorophore from the imaging nucleic acid strand.
Embodiment 176 comprises the method of any one of embodiments 165 to 175, wherein the cleavable moiety is located between the fluorophore and the imaging nucleic acid strand, in the imaging nucleic acid strand, or between a binding molecule and the fluorophore wherein the binding molecule binds to the target molecule and is connected to the imaging nucleic acid strand.
Embodiment 177 comprises the method of any one of embodiments 165 to 175, wherein the cleavable moiety is located within the sequence of the imaging nucleic acid strand.
Embodiment 178 comprises the method of any one of embodiments 165 to 175, wherein the cleavable moiety is located between nucleotides of the imaging nucleic acid strand.
Embodiment 179 comprises the method of any one of embodiments 165 to 175, wherein the cleavable moiety is located in or is part of the fluorophore.
Embodiment 180 comprises the method of any one of embodiments 165 to 175, wherein the imaging nucleic acid strand includes at least two cleavable moieties, and wherein the at least two cleavable moieties are located in the imaging nucleic acid strand, in the fluorophore, or between the binding molecule and the fluorophore.
Embodiment 181 comprises the method of embodiment 180, wherein the at least two cleavable moieties are independently cleavable.
Embodiment 182 comprises the method of any one of embodiments 165 to 181, wherein the fluorophore is located at the 5′-end of the imaging nucleic acid strand.
Embodiment 183 comprises the method of any one of embodiments 165 to 181, wherein the fluorophore is located at the 3′-end of the imaging nucleic acid strand.
Embodiment 184 comprises the method of any one of embodiments 165 to 181, wherein the fluorophore is located between the 5′-end and the 3′-end of the imaging nucleic acid strand.
Embodiment 185 comprises the method of any one of embodiments 165 to 181, wherein the fluorophore is located at or near a middle of the imaging nucleic acid strand.
Embodiment 186 comprises the method of any one of embodiments 165 to 185, wherein the imaging nucleic acid strand comprises deoxyribonucleic acid (DNA).
Embodiment 187 comprises the method of any one of embodiments 165 to 186, wherein the imaging nucleic acid strand comprises peptide nucleic acid (PNA).
Embodiment 188 comprises the method of any one of embodiments 165 to 187, wherein the imaging nucleic acid strand comprises ribonucleic acid (RNA).
Embodiment 189 comprises the method of any one of embodiments 165 to 188, wherein the imaging nucleic acid strand comprises an aptamer.
Embodiment 190 comprises the method of any one of embodiments 165 to 189, wherein the imaging nucleic acid strand comprises one or more non-natural nucleic acids.
Embodiment 191 comprises the method of embodiment 190, wherein the one or more non-natural nucleic acids comprise one or more phosphorothionate linkages.
Embodiment 192 comprises the method of embodiment 190, wherein the one or more non-natural nucleic acids comprise one or more boranophosphate linkages.
Embodiment 193 comprises the method of embodiment 190, wherein the one or more non-natural nucleic acids comprise one or more phosphonate linkages.
Embodiment 194 comprises the method of embodiment 190, wherein the one or more non-natural nucleic acids comprise threose nucleic acid (TNA).
Embodiment 195 comprises the method of embodiment 190, wherein the one or more non-natural nucleic acids comprise locked nucleic acid (LNA).
Embodiment 196 comprises the method of embodiment 190, wherein the one or more non-natural nucleic acids comprise hexitol nucleic acid (HNA).
Embodiment 197 comprises the method of embodiment 190, wherein the one or more non-natural nucleic acids comprise flexible nucleic acid (FNA).
Embodiment 198 comprises the method of embodiment 190, wherein the one or more non-natural nucleic acids comprise glycerol nucleic acid (GNA).
Embodiment 199 comprises the method of embodiment 190, wherein the one or more non-natural nucleic acids comprise a nucleobase analog.
Embodiment 200 comprises the method of any one of embodiments 165 to 199, further comprising fixing the cell sample prior to labeling the target molecule.
Embodiment 201 comprises the method of any one of embodiments 165 to 200, wherein the cell sample comprises disseminated cells.
Embodiment 202 comprises the method of any one of embodiments 165 to 201, wherein the cell sample comprises tissue fragments.
Embodiment 203 comprises the method of any one of embodiments 165 to 202, wherein the cell sample comprises a tissue sample.
Embodiment 204 comprises the method of embodiment 203, wherein the tissue sample is fixed.
Embodiment 205 comprises the method of embodiment 203, wherein the tissue sample is a live tissue sample.
Embodiment 206 comprises the method of any one of embodiments 165 to 200, wherein the cell sample comprises cultured cells.
Embodiment 207 comprises the method of embodiment 206, wherein the cultured cells are fixed.
Embodiment 208 comprises the method of embodiment 206, wherein the cultured cells are live.
Embodiment 209 comprises the method of any one of embodiments 165 to 208, wherein the method is carried out without changing buffer conditions of the cell sample.
Embodiment 210 comprises the method of any one of embodiments 165 to 208, wherein all of the steps of the method are carried out in a same buffer, or wherein buffer conditions and/or concentration of the binding molecule and/or the imaging nucleic acid strand are changed at each step.
Embodiment 211 comprises the method of any one of embodiments 165 to 210, wherein the method is carried out in physiological buffer conditions.
Embodiment 212 comprises the method of any one of embodiments 165 to 208, wherein all of the steps of the method are carried out in a single buffer having a physiological pH and a physiological salt concentration.
Embodiment 213 comprises the method of any one of embodiments 165 to 208, wherein all of the steps of the method are carried out in a single buffer having a pH in a range of about 7.2 to about 7.5, and a sodium chloride concentration in a range of about 135 millimolar (mM) to about 145 mM.
Embodiment 214 comprises the method of any one of embodiments 165 to 208, wherein the method is carried out in a buffer having a sodium chloride concentration in a range of about 135 millimolar (mM) to about 145 mM.
Embodiment 215 comprises the method of any one of embodiments 165 to 208, wherein all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4.
Embodiment 216 comprises the method of any one of embodiments 165 to 208, wherein one or more steps of the method are carried out in a buffer having a salt concentration of about 50 millimolar (mM).
Embodiment 217 comprises the method of any one of embodiments 165 to 216, wherein the cleaving removes the fluorophore from the imaging nucleic strand.
Embodiment 218 comprises the method of any one of embodiments 165 to 217, further comprising washing the cell sample after the cleaving, or diluting the imaging nucleic acid strand or the fluorophore after the cleaving.
Embodiment 219 comprises the method of embodiment 218, further comprising acquiring an SRM or confocal image of the cell sample after the washing to confirm removal of the fluorophore.
Embodiment 220 comprises the method of any one of embodiments 165 to 219, wherein the target molecule comprises a cancer protein.
Embodiment 221 comprises the method of any one of embodiments 165 to 220, wherein the target molecule comprises a tumor antigen.
Embodiment 222 comprises the method of any one of embodiments 165 to 221, wherein the target molecule comprises a ligand.
Embodiment 223 comprises the method of any one of embodiments 165 to 222, wherein the target molecule comprises a lipid.
Embodiment 224 comprises the method of any one of embodiments 165 to 223, wherein the target molecule comprises a carbohydrate.
Embodiment 225 comprises the method of any one of embodiments 165 to 224, wherein labeling the target molecule comprises binding a binding molecule to the target molecule, wherein the binding molecule selectively binds to the target molecule, and wherein the binding molecule is connected to the imaging nucleic acid strand, optionally wherein the at least one cleavable moiety is located between the binding molecule and the fluorophore, and optionally wherein a concentration of the binding molecule is changed at each step.
Embodiment 226 comprises the method of any one of embodiments 165 to 224, wherein labeling the target molecule comprises directly connecting the imaging nucleic acid strand to the target molecule.
Embodiment 227 comprises the method of embodiment 225, wherein labeling the target molecule comprises connecting the imaging nucleic acid strand to the binding molecule by a linker, optionally wherein the at least one cleavable moiety is in the linker, and optionally wherein at least two cleavable moieties are in the linker.
Embodiment 228 comprises the method of embodiments 225 or 227, wherein the binding molecule is selected from an antibody, a vhh antibody, a single-chain variable fragment (scFv), a Fab antibody fragment, a Fab antibody, a secondary antibody, a ligand, and a cofactor.
Embodiment 229 comprises the method of embodiment 228, wherein the ligand comprises a small molecule, a peptide, or a protein.
Embodiment 230 comprises the method of embodiment 228, wherein the binding molecule is a vhh antibody.
Embodiment 231 comprises the method of embodiment 227, wherein the linker comprises a polypeptide.
Embodiment 232 comprises the method of embodiments 227 or 231, wherein the linker connects the N-terminus or C-terminus of the binding molecule with the 5′-end or the 3′-end of the imaging nucleic acid strand.
Embodiment 233 comprises the method of any one of embodiments 225 to 232, wherein the binding molecule and the imaging nucleic acid strand are connected via a sortase-mediated ligation.
Embodiment 234 comprises the method of embodiment 233, wherein the sortase-mediated ligation comprises ligating a C-terminal LPXTG-XX sequence of the binding molecule with a GGG peptide at the 5′-end of the imaging nucleic acid strand.
Embodiment 235 comprises the method of any one of embodiments 225 to 232, wherein the binding molecule is connected to the imaging nucleic acid strand via a side chain of the binding molecule.
Embodiment 236 comprises the method of embodiment 235, wherein the side chain is an arginine side chain or a cysteine side chain.
Embodiment 237 comprises a method of providing a super-resolution microscopy (SRM) image of a target molecule expressed on cell surfaces of a cell sample, comprising: a) binding a binding molecule to the target molecule, wherein the binding molecule is connected to a fluorophore and at least one cleavable moiety, and wherein the binding of the binding molecule to the target molecule brings the fluorophore in proximity to the target molecule; b) imaging the cell sample to provide the SRM image of the target molecule; and c) cleaving the cleavable moiety.
Embodiment 238 comprises the method of embodiment 237, wherein cleaving the cleavable moiety displaces the fluorophore from proximity to the target molecule.
Embodiment 239 comprises the method of embodiments 237 or 238, further comprising providing a multiplexed image of a number (n) of different target molecules expressed on the cell surfaces of the cell sample in a single step or by: repeating steps a) to c) for each of the n different target molecules to provide an SRM image of each of the n different target molecules; and combining the SRM images of the n different target molecules to provide the multiplexed image.
Embodiment 240 comprises the method of any one of embodiments 237 to 239, wherein imaging the cell sample comprises imaging the cell sample by stimulated emission depletion (STED) microscopy, ground state depletion (GSD) microscopy, MINFLUX microscopy, stochastic optical reconstruction microscopy (STORM), direct stochastic optical reconstruction microscopy (DSTORM), or single-molecule localization microscopy.
Embodiment 241 comprises the method of embodiment 239, wherein n is at least 10, n is at least 20, n is at least 50, or n is at least 100.
Embodiment 242 comprises the method of any one of embodiments 237 to 241, wherein cleaving the cleavable moiety comprises cleaving the cleavable moiety with light.
Embodiment 243 comprises the method of any one of embodiments 237 to 242, wherein cleaving the cleavable moiety comprises cleaving the cleavable moiety with UV light.
Embodiment 244 comprises the method of any one of embodiments 237 to 241, wherein cleaving the cleavable moiety comprises cleaving the cleavable moiety via chemical cleavage.
Embodiment 245 comprises the method of any one of embodiments 237 to 241, wherein cleaving the cleavable moiety comprises cleaving the cleavable moiety via enzymatic cleavage.
Embodiment 246 comprises the method of any one of embodiments 237 to 241, wherein cleaving the cleavable moiety comprises cleaving the cleavable moiety with a nuclease.
Embodiment 247 comprises the method of any one of embodiments 237 to 246, wherein the cleavable moiety is located in or is part of the fluorophore.
Embodiment 248 comprises the method of any one of embodiments 237 to 247, further comprising fixing the cell sample prior to labeling the target molecule.
Embodiment 249 comprises the method of any one of embodiments 237 to 248, wherein the cell sample comprises disseminated cells.
Embodiment 250 comprises the method of any one of embodiments 237 to 249, wherein the cell sample comprises tissue fragments.
Embodiment 251 comprises the method of any one of embodiments 237 to 250, wherein the cell sample comprises a tissue sample.
Embodiment 252 comprises the method of embodiment 251, wherein the tissue sample is fixed.
Embodiment 253 comprises the method of embodiment 251, wherein the tissue sample is a live tissue sample.
Embodiment 254 comprises the method of any one of embodiments 237 to 247, wherein the cell sample comprises cultured cells.
Embodiment 255 comprises the method of embodiment 254, wherein the cultured cells are fixed.
Embodiment 256 comprises the method of embodiment 254, wherein the cultured cells are live.
Embodiment 257 comprises the method of any one of embodiments 237 to 256, wherein the method is carried out without changing buffer conditions of the cell sample.
Embodiment 258 comprises the method of any one of embodiments 237 to 256, wherein all of the steps of the method are carried out in a same buffer, or wherein buffer conditions are changed at each step.
Embodiment 259 comprises the method of any one of embodiments 237 to 258, wherein the method is carried out in physiological buffer conditions.
Embodiment 260 comprises the method of any one of embodiments 237 to 258, wherein all of the steps of the method are carried out in a single buffer having a physiological pH and a physiological salt concentration.
Embodiment 261 comprises the method of any one of embodiments 237 to 258, wherein all of the steps of the method are carried out in a single buffer having a pH in a range of about 7.2 to about 7.5, and a sodium chloride concentration in a range of about 135 millimolar (mM) to about 145 mM.
Embodiment 262 comprises the method of any one of embodiments 237 to 258, wherein the method is carried out in a buffer having a sodium chloride concentration in a range of about 135 millimolar (mM) to about 145 mM.
Embodiment 263 comprises the method of any one of embodiments 237 to 258, wherein all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4.
Embodiment 264 comprises the method of any one of embodiments 237 to 258, wherein one or more steps of the method are carried out in a buffer having a salt concentration of about 50 millimolar (mM).
Embodiment 265 comprises the method of any one of embodiments 237 to 264, wherein the cleaving removes the fluorophore from the binding molecule.
Embodiment 266 comprises the method of any one of embodiments 237 to 265, further comprising washing the cell sample after the cleaving, or diluting the fluorophore with buffer after the cleaving.
Embodiment 267 comprises the method of embodiment 266, further comprising acquiring an SRM or confocal image of the cell sample after the washing to confirm removal of the fluorophore.
Embodiment 268 comprises the method of any one of embodiments 237 to 267, wherein the target molecule comprises a cancer protein.
Embodiment 269 comprises the method of any one of embodiments 237 to 268, wherein the target molecule comprises a tumor antigen.
Embodiment 270 comprises the method of any one of embodiments 237 to 269, wherein the target molecule comprises a ligand.
Embodiment 271 comprises the method of any one of embodiments 237 to 270, wherein the target molecule comprises a lipid.
Embodiment 272 comprises the method of any one of embodiments 237 to 271, wherein the target molecule comprises a carbohydrate.
Embodiment 273 comprises the method of any one of embodiments 237 to 272, wherein the binding molecule is selected from an antibody, a vhh antibody, a single-chain variable fragment (scFv), a Fab antibody fragment, a Fab antibody, a secondary antibody, a ligand, and a cofactor.
Embodiment 274 comprises the method of embodiment 273, wherein the ligand comprises a small molecule, a peptide, or a protein.
Embodiment 275 comprises the method of embodiment 273, wherein the binding molecule is a vhh antibody.
Embodiment 276 comprises the method of any one of embodiments 237 to 275, wherein the binding molecule is connected to the cleavable moiety by a linker, optionally wherein the cleavable moiety is located in the linker.
Embodiment 277 comprises the method of any one of embodiments 237 to 276, wherein the binding molecule is connected to the cleavable moiety via a sortase-mediated ligation.
Embodiment 278 comprises the method of any one of embodiments 237 to 276, wherein the binding molecule is connected to the cleavable moiety via a side chain of the binding molecule.
Embodiment 279 comprises the method of embodiment 278, wherein the side chain is an arginine side chain or a cysteine side chain.
Embodiment 280 comprises the method of any one of embodiments 237 to 279, wherein the binding molecule is connected to at least two cleavable moieties, optionally wherein the at least two cleavable moieties are located in the fluorophore, between the binding molecule and the fluorophore, or in the linker.
Embodiment 281 comprises the method of any one of embodiments 237 to 280, wherein the cleavable moiety is located between the binding molecule and the fluorophore or is located in or is part of the fluorophore.

### EXAMPLES

### Example 1: Exemplary Method for DNA-Based Super Resolution Application using STED Imaging

Reagents and materials for the experiment will be obtained. These reagents and materials will include:
cells or tissue on a #1.5 glass cover slip;
15 different VHHs with C-terminal sortase tag;
15 peptide-oligo conjugates: Gly-Gly-Gly-(docking strand oligo);
attach a different docking strand to each of the different VHHs using the sortase-mediated ligation reaction followed by purifying each thus labeled VHH;
15 imaging strands consisting of fluorescently labeled oligos complementary to the docking strands that include a UV cleavable linker between the oligo and the fluorophore. For spectral multiplexing use 3 fluorophores, i.e. the 1^{st}, 4^{th}, 7^{th}, 10^{th}, and 13^{th} imaging strands use a red fluorophore, the 2^{nd}, 5^{th}, 8^{th}, 11^{th}, and 14^{th} an orange fluorophore, and the 3^{rd}, 6^{th}, 9^{th}, 12^{th}, and 15^{th} use a green fluorophore;
wash buffer consisting of PBS;
hybridization buffer consisting of PBS with 500 mM NaCl, 10 mM magnesium chloride (MgCl₂); and
imaging buffer consisting of hybridization buffer and antifade to suppress photobleaching.

Cells or tissues will be stained using a mixture of all VHHs labeled with docking strands. Excess unbound VHHs will be washed away. Cells (or tissue) will be fixed using formaldehyde, and any excess formaldehyde will be washed away.

The #1.5 glass coverslip with cells (or tissue) will be mounted in a flow cell that is placed on the super resolution microscope, and the flow cell will be equilibrated with hybridization buffer. The first 3 imaging strands will be flowed into the flow cell in hybridization buffer allowing hybridization to occur for 20 minutes. Following hybridization, imaging buffer will be flowed into the flow cell to wash away any unbound imaging strands. A STED image of the sample will be acquired using the SRM. The cells (or tissue) will be exposed to UV light to cleave the fluorophores from the imaging strands while flowing hybridization buffer through the flow cell.

Alternatively, wash buffer and/or unlabeled imaging strands (without fluorophore and cleavable linker) can be used to dissociate imaging and docking strands and remove the fluorophores while exposing the sample to UV light, followed by equilibration with hybridization buffer. A low resolution (confocal) image of a region of the sample that had a bright signal will be obtained to ensure all fluorophore is removed. The next 3 imaging strands will be flowed into the flow cell in hybridization buffer allowing hybridization to occur for 20 minutes, and the steps of washing, flowing in imaging buffer, acquiring a STED image, exposing the cells (or tissue) to UV light, and ensuring removal of the fluorophore will be repeated until all imaging strands have been used up in successive rounds of imaging.

The acquired images will be overlaid and a drift correction will be applied to bring these into register. The images will be analyzed.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method of providing a super-resolution microscopy (SRM) image of a target molecule expressed on cell surfaces of a cell sample, comprising:
a) labeling the target molecule with a docking nucleic acid strand;
b) hybridizing an imaging nucleic acid strand to the docking nucleic acid strand, wherein the imaging nucleic acid strand includes a fluorophore and at least one cleavable moiety, and wherein the hybridizing brings the fluorophore in proximity to the target molecule;
c) imaging the cell sample to provide the SRM image of the target molecule; and
d) cleaving the cleavable moiety to displace the fluorophore from proximity to the target molecule, to change a blinking behavior of the imaging nucleic acid strand, and/or to change a binding specificity of the imaging nucleic acid strand.

2. The method of claim 1, wherein cleaving the cleavable moiety displaces the fluorophore from proximity to the target molecule.

3. The method of claim 1, further comprising providing a multiplexed image of a number (n) of different target molecules expressed on the cell surfaces of the cell sample by:
repeating steps a) to d) for each of the n different target molecules to provide an SRM image of each of the n different target molecules; and
combining the SRM images of the n different target molecules to provide the multiplexed image.

4. A method of providing a multiplexed super-resolution microscopy (SRM) image of a number (n) of different target molecules expressed on cell surfaces of a cell sample, comprising:
i) labeling each of the different target molecules with a different docking nucleic acid strand;
ii) obtaining a SRM image of at least one target molecule of the n different target molecules by:
hybridizing an imaging nucleic acid strand to the docking nucleic acid strand associated with the target molecule, wherein the imaging nucleic acid strand includes a fluorophore and a cleavable moiety, and wherein the hybridizing brings the fluorophore of the imaging nucleic acid strand in proximity to the target molecule,
imaging the cell sample using fluorescence emission of the fluorophore, and
cleaving the cleavable moiety to remove the fluorophore from proximity to the target molecule, to change a blinking behavior of the imaging nucleic acid strand, and/or to change a binding specificity of the imaging nucleic acid strand;
iii) repeating step ii) until SRM images of all of the n different target molecules of the cell sample are obtained; and
iv) combining the SRM images of all of the n different target molecules to provide the multiplexed SRM image of the n different target molecules expressed on the cell surfaces of the cell sample.

5. The method of claim 1, wherein imaging the cell sample comprises imaging the cell sample by stimulated emission depletion (STED) microscopy, ground state depletion (GSD) microscopy, MINFLUX microscopy, stochastic optical reconstruction microscopy (STORM), direct stochastic optical reconstruction microscopy (DSTORM), or single-molecule localization microscopy.

6. The method of claim 3, wherein a different imaging nucleic acid strand is used for imaging each of the n different target molecules.

7. The method of claim 3, wherein one imaging nucleic acid strand is used to image a subset of two or more of the n different target molecules.

8. The method of claim 4, wherein the step of ii) obtaining the SRM image comprises obtaining an SRM image of a subset of target molecules of the n different target molecules simultaneously.

9. The method of claim 8, wherein a different imaging nucleic acid strand with a different fluorophore is used for imaging each of the target molecules in the subset.

10. The method of claim 8, wherein the subset includes at least 2 different target molecules.

11. The method of claim 8, wherein the subset includes at least 3 different target molecules.

12. The method of claim 3, wherein n is at least 10.

13. The method of claim 3, wherein n is at least 20.

14. The method of claim 3, wherein n is at least 50.

15. The method of claim 3, wherein n is at least 100.

16. The method of claim 1, wherein cleaving the cleavable moiety comprises cleaving the cleavable moiety with light.

17. The method of claim 1, wherein cleaving the cleavable moiety comprises cleaving the cleavable moiety with UV light.

18. The method of claim 1, wherein cleaving the cleavable moiety comprises cleaving the cleavable moiety via chemical cleavage.

19. The method of claim 1, wherein cleaving the cleavable moiety comprises cleaving the cleavable moiety via enzymatic cleavage.

20. The method of claim 1, wherein cleaving the cleavable moiety comprises cleaving the cleavable moiety with a nuclease.

21. The method of claim 1, wherein cleaving the cleavable moiety releases the fluorophore from the imaging nucleic acid strand.

22. The method of claim 1, wherein cleaving the cleavable moiety releases at least a portion of the imaging nucleic acid strand from the docking nucleic acid strand.

23. The method of claim 1, further comprising displacing the imaging nucleic acid strand from the docking nucleic acid strand by hybridizing a non-labeled nucleic acid strand to the docking nucleic acid strand after cleaving the cleavable moiety.

24. The method of claim 1, wherein cleaving the cleavable moiety releases the imaging nucleic acid strand from the docking nucleic acid strand.

25. The method of claim 1, wherein cleaving the cleavable moiety changes kinetics of dissociation of the imaging nucleic acid strand from the docking nucleic acid strand, thereby changing the blinking behavior of the imaging nucleic acid strand.

26. The method of claim 1, wherein cleaving the cleavable moiety changes the binding specificity of the imaging nucleic acid strand for the docking nucleic acid strand.

27. The method of claim 1, wherein: the cleavable moiety is located between the fluorophore and the imaging nucleic acid strand, the cleavable moiety is located in or is part of the fluorophore, the cleavable moiety is in the docking nucleic acid strand, the cleavable moiety is between a binding molecule and the fluorophore wherein the binding molecule binds to the target molecule and is connected to the docking nucleic acid strand, or the cleavable moiety is in the imaging nucleic acid strand.

28. The method of claim 1, wherein the cleavable moiety is located within the sequence of the imaging nucleic acid strand.

29. The method of claim 1, wherein the cleavable moiety is located between nucleotides of the imaging nucleic acid strand.

30. The method of claim 27, wherein the imaging nucleic acid strand includes at least two cleavable moieties, and optionally wherein the at least two cleavable moieties are located in the imaging nucleic acid strand, in the docking nucleic acid strand, between the binding molecule and the fluorophore, or in the fluorophore.

31. The method of claim 1, wherein the imaging nucleic acid strand includes at least two cleavable moieties that are independently cleavable.

32. The method of claim 1, wherein the hybridizing brings the fluorophore within a distance of about 2 nanometers (nm) to about 10 nm from the target molecule.

33. The method of claim 1, wherein the fluorophore is located at the 5′-end of the imaging nucleic acid strand.

34. The method of claim 1, wherein the fluorophore is located at the 3′-end of the imaging nucleic acid strand.

35. The method of claim 1, wherein the fluorophore is located between the 5′-end and the 3′-end of the imaging nucleic acid strand.

36. The method of claim 1, wherein the fluorophore is located at or near a middle of the imaging nucleic acid strand.

37. The method of claim 1, wherein the imaging nucleic acid strand comprises deoxyribonucleic acid (DNA).

38. The method of claim 1, wherein the imaging nucleic acid strand comprises peptide nucleic acid (PNA).

39. The method of claim 1, wherein the docking nucleic acid strand comprises deoxyribonucleic acid (DNA).

40. The method of claim 1, wherein the docking nucleic acid strand comprises peptide nucleic acid (PNA).

41. The method of claim 1, wherein one or both of the docking nucleic acid strand and the imaging nucleic acid strand comprise ribonucleic acid (RNA).

42. The method of claim 1, wherein one or both of the docking nucleic acid strand and the imaging nucleic acid strand comprise an aptamer.

43. The method of claim 1, wherein one or both of the docking nucleic acid strand and the imaging nucleic acid strand comprise one or more non-natural nucleic acids.

44. The method of claim 43, wherein the one or more non-natural nucleic acids comprise one or more phosphorothionate backbone linkages.

45. The method of claim 43, wherein the one or more non-natural nucleic acids comprise one or more boranophosphate backbone linkages.

46. The method of claim 43, wherein the one or more non-natural nucleic acids comprise one or more phosphonate backbone linkages.

47. The method of claim 43, wherein the one or more non-natural nucleic acids comprise threose nucleic acid (TNA).

48. The method of claim 43, wherein the one or more non-natural nucleic acids comprise locked nucleic acid (LNA).

49. The method of claim 43, wherein the one or more non-natural nucleic acids comprise hexitol nucleic acid (HNA).

50. The method of claim 43, wherein the one or more non-natural nucleic acids comprise flexible nucleic acid (FNA).

51. The method of claim 43, wherein the one or more non-natural nucleic acids comprise glycerol nucleic acid (GNA).

52. The method of claim 43, wherein the one or more non-natural nucleic acids comprise a nucleobase analog.

53. The method of claim 1, further comprising fixing the cell sample prior to labeling the target molecule.

54. The method of claim 1, wherein the cell sample comprises disseminated cells.

55. The method of claim 1, wherein the cell sample comprises tissue fragments.

56. The method of claim 1, wherein the cell sample comprises a tissue sample.

57. The method of claim 56, wherein the tissue sample is fixed.

58. The method of claim 56, wherein the tissue sample is a live tissue sample.

59. The method of claim 1, wherein the cell sample comprises cultured cells.

60. The method of claim 59, wherein the cultured cells are fixed.

61. The method of claim 59, wherein the cultured cells are live.

62. The method of claim 1, wherein the method is carried out without changing buffer conditions of the cell sample.

63. The method of claim 27, wherein all of the steps of the method are carried out in a same buffer, or wherein buffer conditions and/or concentration of the binding molecule and/or the imaging nucleic acid strand is changed at each step.

64. The method of claim 1, wherein the method is carried out in physiological buffer conditions.

65. The method of claim 1, wherein all of the steps of the method are carried out in a single buffer having a physiological pH and a physiological salt concentration.

66. The method of claim 1, wherein all of the steps of the method are carried out in a single buffer having a pH in a range of about 7.2 to about 7.5, and a sodium chloride concentration in a range of about 135 millimolar (mM) to about 145 mM.

67. The method of claim 1, wherein the method is carried out in a buffer having a sodium chloride concentration in a range of about 135 millimolar (mM) to about 145 mM.

68. The method of claim 1, wherein all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4.

69. The method of claim 1, wherein one or more steps of the method are carried out in a buffer having a salt concentration of about 500 millimolar (mM).

70. The method of claim 1, wherein one or more of the steps of the method are carried out in a buffer having a salt concentration in a range of about 500 millimolar (mM) to about 600 mM.

71. The method of claim 1, wherein one or more steps of the method are carried out in a buffer having a salt concentration of about 50 millimolar (mM).

72. The method of claim 1, wherein the cleaving removes the fluorophore from the imaging nucleic acid strand.

73. The method of claim 72, further comprising washing the cell sample after the cleaving, or diluting the docking nucleic acid strand or the imaging nucleic acid strand or the fluorophore with buffer after the cleaving.

74. The method of claim 73, further comprising acquiring an SRM or confocal image of the cell sample after the washing to confirm removal of the fluorophore.

75. The method of claim 1, wherein the cleaving causes the imaging nucleic acid strand to dissociate from the docking nucleic acid strand.

76. The method of claim 1, wherein the cleaving causes the imaging nucleic acid strand to expose a different sequence of nucleotide that binds to a different docking nucleic acid strand.

77. The method of claim 1, wherein the cleaving removes the fluorophore and at least a portion of the imaging nucleic acid strand from the docking nucleic acid strand.

78. The method of claim 77, further comprising washing the cell sample after the cleaving.

79. The method of claim 78, further comprising acquiring an SRM or confocal image of the cell sample after the washing to confirm removal of the fluorophore and the imaging nucleic acid strand from the docking nucleic acid strand.

80. The method of claim 1, wherein the hybridizing results in a stable complex being formed between imaging nucleic acid strand and the docking nucleic acid strand.

81. The method of claim 80, wherein the stable complex has a lifetime of at least about 1 second.

82. The method of claim 80, wherein the stable complex has a lifetime of at least about 1 hour.

83. The method of claim 80, wherein the stable complex has a lifetime of more than 1 hour.

84. The method of claim 80, wherein the imaging nucleic acid strand exhibits non-blinking behavior due to the stable complex formed between the imaging nucleic acid strand and the docking nucleic acid strand.

85. The method of claim 80, wherein the cleaving destabilizes the stable complex formed between the imaging nucleic acid strand and the docking nucleic acid strand, thereby causing the imaging nucleic acid strand and the docking nucleic acid strand to form a transient complex, and causing the imaging nucleic acid strand to exhibit blinking behavior.

86. The method of claim 80, wherein the cleaving causes the imaging nucleic acid strand to exhibit a blinking behavior.

87. The method of claim 1, wherein the hybridizing results in a transient complex being formed between the imaging nucleic acid strand and the docking nucleic acid strand.

88. The method of claim 87, wherein the imaging nucleic acid strand exhibits blinking behavior due to the transient complex formed between the imaging nucleic acid strand and the docking nucleic acid strand.

89. The method of claim 87, wherein the cleaving modulates binding properties of the transient complex, resulting in a change in frequency and/or duration of blinking of the imaging nucleic acid strand.

90. The method of claim 87, wherein the cleaving modulates binding properties of the transient complex, causing the imaging nucleic acid strand to bind to a docking nucleic acid strand of another target molecule.

91. The method of claim 88, wherein kinetics of the blinking behavior is correlated with a concentration of the imaging nucleic acid strand.

92. The method of claim 1, wherein the target molecule comprises a cancer protein.

93. The method of claim 1, wherein the target molecule comprises a tumor antigen.

94. The method of claim 1, wherein the target molecule comprises a ligand.

95. The method of claim 1, wherein the target molecule comprises a lipid.

96. The method of claim 1, wherein the target molecule comprises a carbohydrate.

97. The method of claim 1, wherein labeling the target molecule comprises binding a binding molecule to the target molecule, wherein the binding molecule selectively binds to the target molecule, and wherein the binding molecule is connected to the docking nucleic acid strand, optionally wherein the cleavable moiety is located between the binding molecule and the fluorophore, and optionally wherein a concentration of the binding molecule is changed at each step.

98. The method of claim 1, wherein labeling the target molecule comprises directly connecting the docking nucleic acid strand to the target molecule.

99. The method of claim 97, wherein labeling the target molecule comprises connecting the docking nucleic acid strand to the binding molecule by a linker, optionally wherein the at least one cleavable moiety is located between the binding molecule and the docking nucleic acid strand or in the linker, optionally wherein the at least one cleavable moiety is located between the binding molecule and the imaging nucleic acid strand or in the linker, or optionally wherein at least two cleavable moieties are in the linker.

100. The method of claim 97, wherein the binding molecule is selected from an antibody, a vhh antibody, a single-chain variable fragment (scFv), a Fab antibody fragment, a Fab antibody, a secondary antibody, a ligand, and a cofactor.

101. The method of claim 100, wherein the ligand comprises a small molecule, a peptide, or a protein.

102. The method of claim 100, wherein the binding molecule is a vhh antibody.

103. The method of claim 99, wherein the linker comprises a polypeptide.

104. The method of claim 99, wherein the linker connects the N-terminus or C-terminus of the binding molecule with the 5′-end or the 3′-end of the docking nucleic acid strand.

105. The method of claim 99, wherein the binding molecule and the docking nucleic acid strand are connected via a sortase-mediated ligation.

106. The method of claim 105, wherein the sortase-mediated ligation comprises ligating a C-terminal LPXTG-XX sequence of the binding molecule with a GGG peptide at the 5′-end of the docking nucleic acid strand.

107. The method of claim 97, wherein the binding molecule is connected to the docking nucleic acid strand via a side chain of the binding molecule.

108. The method of claim 107, wherein the side chain is an arginine side chain or a cysteine side chain.

109. A cell sample for super-resolution microscopy (SRM) imaging, comprising:
a target molecule expressed on cell surfaces of the cell sample;
a docking nucleic acid strand associated with the target molecule; and
an imaging nucleic acid strand hybridized to the docking nucleic acid strand, wherein the imaging nucleic acid strand includes a fluorophore and a cleavable moiety.

110. The cell sample of claim 109, wherein the cleavable moiety is cleavable with light.

111. The cell sample of claim 109, wherein the cleavable moiety is cleavable with UV light.

112. The cell sample of claim 109, wherein the cleavable moiety is cleavable by chemical cleavage.

113. The cell sample of claim 109, wherein the cleavable moiety is cleavable by enzymatic cleavage.

114. The cell sample of claim 109, wherein the cleavable moiety is cleavable with a nuclease.

115. The cell sample of claim 109, wherein: the cleavable moiety is located between the fluorophore and the imaging nucleic acid strand, the cleavable moiety is located in or is part of the fluorophore, the cleavable moiety is in the docking nucleic acid strand, the cleavable moiety is between a binding molecule and the fluorophore wherein the binding molecule binds to the target molecule and is connected to the docking nucleic acid strand, or the cleavable moiety is in the imaging nucleic acid strand.

116. The cell sample of claim 109, wherein the cleavable moiety is located within the sequence of the imaging nucleic acid strand.

117. The cell sample of claim 109, wherein the cleavable moiety is located between nucleotides of the imaging nucleic acid strand.

118. The cell sample of claim 115, wherein the imaging nucleic acid strand includes at least two cleavable moieties, and optionally wherein the at least two cleavable moieties are located in the imaging nucleic acid strand, in the docking nucleic acid strand, between the binding molecule and the fluorophore, or in the fluorophore.

119. The cell sample of claim 118, wherein the at least two cleavable moieties are independently cleavable.

120. The cell sample of claim 109, wherein the fluorophore is located at the 5′-end of the imaging nucleic acid strand.

121. The cell sample of claim 109, wherein the fluorophore is located at the 3′-end of the imaging nucleic acid strand.

122. The cell sample of claim 109, wherein the fluorophore is located between the 5′-end and the 3′-end of the imaging nucleic acid strand.

123. The cell sample of claim 109, wherein the fluorophore is located at or near a middle of the imaging nucleic acid strand.

124. The cell sample of claim 109, wherein the imaging nucleic acid strand comprises deoxyribonucleic acid (DNA).

125. The cell sample of claim 109, wherein the imaging nucleic acid strand comprises peptide nucleic acid (PNA).

126. The cell sample of claim 109, wherein the docking nucleic acid strand comprises deoxyribonucleic acid (DNA).

127. The cell sample of claim 109, wherein the docking nucleic acid strand comprises peptide nucleic acid (PNA).

128. The cell sample of claim 109, wherein one or both of the docking nucleic acid strand and the imaging nucleic acid strand comprises ribonucleic acid (RNA).

129. The cell sample of claim 109, wherein one or both of the docking nucleic acid strand and the imaging nucleic acid strand comprise an aptamer.

130. The cell sample of claim 109, wherein one or both of the docking nucleic acid strand and the imaging nucleic acid strand comprise one or more non-natural nucleic acids.

131. The cell sample of claim 130, wherein the one or more non-natural nucleic acids comprise one or more phosphorothionate linkages.

132. The cell sample of claim 130, wherein the one or more non-natural nucleic acids comprise one or more boranophosphate linkages.

133. The cell sample of claim 130, wherein the one or more non-natural nucleic acids comprise one or more phosphonate linkages.

134. The cell sample of claim 130, wherein the one or more non-natural nucleic acids comprise threose nucleic acid (TNA).

135. The cell sample of claim 130, wherein the one or more non-natural nucleic acids comprise locked nucleic acid (LNA).

136. The cell sample of claim 130, wherein the one or more non-natural nucleic acids comprise hexitol nucleic acid (HNA).

137. The cell sample of claim 130, wherein the one or more non-natural nucleic acids comprise flexible nucleic acid (FNA).

138. The cell sample of claim 130, wherein the one or more non-natural nucleic acids comprise glycerol nucleic acid (GNA).

139. The cell sample of claim 130, wherein the one or more non-natural nucleic acids comprise a nucleobase analog.

140. The cell sample of claim 109, wherein the cell sample comprises a tissue sample.

141. The cell sample of claim 140, wherein the tissue sample is fixed.

142. The cell sample of claim 140, wherein the tissue sample is a live tissue sample.

143. The cell sample of claim 109, wherein the cell sample comprises cultured cells.

144. The cell sample of claim 143, wherein the cultured cells are fixed.

145. The cell sample of claim 143, wherein the cultured cells are live.

146. The cell sample of claim 109, wherein the cell sample comprises disseminated cells.

147. The cell sample of claim 109, wherein the cell sample comprises tissue fragments.

148. The cell sample of claim 109, wherein the docking nucleic acid strand and the imaging nucleic acid strand form a stable complex.

149. The cell sample of claim 148, wherein the stable complex has a lifetime of at least about 1 second.

150. The cell sample of claim 148, wherein the stable complex has a lifetime of at least about 1 hour.

151. The cell sample of claim 148, wherein the stable complex has a lifetime of more than 1 hour.

152. The cell sample of claim 109, wherein the target molecule comprises a cancer protein.

153. The cell sample of claim 109, wherein the target molecule comprises a tumor antigen.

154. The cell sample of claim 109, wherein the target molecule comprises a ligand.

155. The cell sample of claim 109, wherein the target molecule comprises a lipid.

156. The cell sample of claim 109, wherein the target molecule comprises a carbohydrate.

157. The cell sample of claim 109, wherein the target molecule is directly connected to the docking nucleic acid strand.

158. The cell sample of claim 109, wherein the docking nucleic acid strand is connected to a binding molecule by a linker, and wherein the binding molecule is selectively bound to the target molecule, optionally wherein the at least one cleavable moiety is located between the binding molecule and the docking nucleic acid strand or in the linker, optionally wherein the cleavable moiety is located between the binding molecule and the fluorophore, and optionally wherein at least two cleavable moieties are in the linker.

159. The cell sample of claim 158, wherein the binding molecule is selected from an antibody, a vhh antibody, a single-chain variable fragment (scFv), a Fab antibody fragment, a Fab antibody, a secondary antibody, a ligand, and a cofactor.

160. The cell sample of claim 159, wherein the ligand comprises a small molecule, a peptide, or a protein.

161. The cell sample of claim 159, wherein the binding molecule is a vhh antibody.

162. The cell sample of claim 158, wherein the linker comprises a polypeptide.

163. The cell sample of any one of claim 158, wherein the binding molecule is connected to the docking nucleic acid strand via a side chain of the binding molecule.

164. The cell sample of claim 163, wherein the side chain is an arginine side chain or a cysteine side chain.

165. A method of providing a super-resolution microscopy (SRM) image of a target molecule expressed on cell surfaces of a cell sample, comprising:
a) labeling the target molecule with an imaging nucleic acid strand, wherein the imaging nucleic acid strand includes a fluorophore and at least one cleavable moiety, and wherein the labeling of the target molecule brings the fluorophore in proximity to the target molecule;
b) imaging the cell sample to provide the SRM image of the target molecule; and
c) cleaving the cleavable moiety.

166. The method of claim 165, wherein cleaving the cleavable moiety displaces the fluorophore from proximity to the target molecule.

167. The method of claim 165, further comprising providing a multiplexed image of a number (n) of different target molecules expressed on the cell surfaces of the cell sample in a single step or by: repeating steps a) to c) for each of the n different target molecules to provide an SRM image of each of the n different target molecules; and combining the SRM images of the n different target molecules to provide the multiplexed image.

168. The method of claim 165, wherein imaging the cell sample comprises imaging the cell sample by stimulated emission depletion (STED) microscopy, ground state depletion (GSD) microscopy, MINFLUX microscopy, stochastic optical reconstruction microscopy (STORM), direct stochastic optical reconstruction microscopy (DSTORM), or single-molecule localization microscopy.

169. The method of claim 167, wherein n is at least 10, n is at least 20, n is at least 50, or nis at least 100.

170. The method of claim 165, wherein cleaving the cleavable moiety comprises cleaving the cleavable moiety with light.

171. The method of claim 165, wherein cleaving the cleavable moiety comprises cleaving the cleavable moiety with UV light.

172. The method of claim 165, wherein cleaving the cleavable moiety comprises cleaving the cleavable moiety via chemical cleavage.

173. The method of claim 165, wherein cleaving the cleavable moiety comprises cleaving the cleavable moiety via enzymatic cleavage.

174. The method of claim 165, wherein cleaving the cleavable moiety comprises cleaving the cleavable moiety with a nuclease.

175. The method of claim 165, wherein cleaving the cleavable moiety releases the fluorophore from the imaging nucleic acid strand.

176. The method of claim 165, wherein the cleavable moiety is located between the fluorophore and the imaging nucleic acid strand, in the imaging nucleic acid strand, or between a binding molecule and the fluorophore wherein the binding molecule binds to the target molecule and is connected to the imaging nucleic acid strand.

177. The method of claim 165, wherein the cleavable moiety is located within the sequence of the imaging nucleic acid strand.

178. The method of claim 165, wherein the cleavable moiety is located between nucleotides of the imaging nucleic acid strand.

179. The method of claim 165, wherein the cleavable moiety is located in or is part of the fluorophore.

180. The method of claim 176, wherein the imaging nucleic acid strand includes at least two cleavable moieties, and wherein the at least two cleavable moieties are located in the imaging nucleic acid strand, in the fluorophore, or between the binding molecule and the fluorophore.

181. The method of claim 180, wherein the at least two cleavable moieties are independently cleavable.

182. The method of claim 165, wherein the fluorophore is located at the 5′-end of the imaging nucleic acid strand.

183. The method of claim 165, wherein the fluorophore is located at the 3′-end of the imaging nucleic acid strand.

184. The method of claim 165, wherein the fluorophore is located between the 5′-end and the 3′-end of the imaging nucleic acid strand.

185. The method of claim 165, wherein the fluorophore is located at or near a middle of the imaging nucleic acid strand.

186. The method of claim 165, wherein the imaging nucleic acid strand comprises deoxyribonucleic acid (DNA).

187. The method of claim 165, wherein the imaging nucleic acid strand comprises peptide nucleic acid (PNA).

188. The method of claim 165, wherein the imaging nucleic acid strand comprises ribonucleic acid (RNA).

189. The method of claim 165, wherein the imaging nucleic acid strand comprises an aptamer.

190. The method of claim 165, wherein the imaging nucleic acid strand comprises one or more non-natural nucleic acids.

191. The method of claim 190, wherein the one or more non-natural nucleic acids comprise one or more phosphorothionate linkages.

192. The method of claim 190, wherein the one or more non-natural nucleic acids comprise one or more boranophosphate linkages.

193. The method of claim 190, wherein the one or more non-natural nucleic acids comprise one or more phosphonate linkages.

194. The method of claim 190, wherein the one or more non-natural nucleic acids comprise threose nucleic acid (TNA).

195. The method of claim 190, wherein the one or more non-natural nucleic acids comprise locked nucleic acid (LNA).

196. The method of claim 190, wherein the one or more non-natural nucleic acids comprise hexitol nucleic acid (HNA).

197. The method of claim 190, wherein the one or more non-natural nucleic acids comprise flexible nucleic acid (FNA).

198. The method of claim 190, wherein the one or more non-natural nucleic acids comprise glycerol nucleic acid (GNA).

199. The method of claim 190, wherein the one or more non-natural nucleic acids comprise a nucleobase analog.

200. The method of claim 165, further comprising fixing the cell sample prior to labeling the target molecule.

201. The method of claim 165, wherein the cell sample comprises disseminated cells.

202. The method of claim 165, wherein the cell sample comprises tissue fragments.

203. The method of claim 165, wherein the cell sample comprises a tissue sample.

204. The method of claim 203, wherein the tissue sample is fixed.

205. The method of claim 203, wherein the tissue sample is a live tissue sample.

206. The method of claim 165, wherein the cell sample comprises cultured cells.

207. The method of claim 206, wherein the cultured cells are fixed.

208. The method of claim 206, wherein the cultured cells are live.

209. The method of claim 165, wherein the method is carried out without changing buffer conditions of the cell sample.

210. The method of claim 165, wherein all of the steps of the method are carried out in a same buffer, or wherein buffer conditions and/or concentration of the binding molecule and/or the imaging nucleic acid strand are changed at each step.

211. The method of claim 165, wherein the method is carried out in physiological buffer conditions.

212. The method of claim 165, wherein all of the steps of the method are carried out in a single buffer having a physiological pH and a physiological salt concentration.

213. The method of claim 165, wherein all of the steps of the method are carried out in a single buffer having a pH in a range of about 7.2 to about 7.5, and a sodium chloride concentration in a range of about 135 millimolar (mM) to about 145 mM.

214. The method of claim 165, wherein the method is carried out in a buffer having a sodium chloride concentration in a range of about 135 millimolar (mM) to about 145 mM.

215. The method of claim 165, wherein all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4.

216. The method of claim 165, wherein one or more steps of the method are carried out in a buffer having a salt concentration of about 50 millimolar (mM).

217. The method of claim 165, wherein the cleaving removes the fluorophore from the imaging nucleic strand.

218. The method of claim 165, further comprising washing the cell sample after the cleaving, or diluting the imaging nucleic acid strand or the fluorophore after the cleaving.

219. The method of claim 218, further comprising acquiring an SRM or confocal image of the cell sample after the washing to confirm removal of the fluorophore.

220. The method of claim 165, wherein the target molecule comprises a cancer protein.

221. The method of claim 165, wherein the target molecule comprises a tumor antigen.

222. The method of claim 165, wherein the target molecule comprises a ligand.

223. The method of claim 165, wherein the target molecule comprises a lipid.

224. The method of claim 165, wherein the target molecule comprises a carbohydrate.

225. The method of claim 165, wherein labeling the target molecule comprises binding a binding molecule to the target molecule, wherein the binding molecule selectively binds to the target molecule, and wherein the binding molecule is connected to the imaging nucleic acid strand, optionally wherein the at least one cleavable moiety is located between the binding molecule and the fluorophore, and optionally wherein a concentration of the binding molecule is changed at each step.

226. The method of claim 165, wherein labeling the target molecule comprises directly connecting the imaging nucleic acid strand to the target molecule.

227. The method of claim 225, wherein labeling the target molecule comprises connecting the imaging nucleic acid strand to the binding molecule by a linker, optionally wherein the at least one cleavable moiety is in the linker, optionally wherein the at least one cleavable moiety is located between the binding molecule and the imaging nucleic acid strand or in the linker, and optionally wherein at least two cleavable moieties are in the linker.

228. The method of claim 225, wherein the binding molecule is selected from an antibody, a vhh antibody, a single-chain variable fragment (scFv), a Fab antibody fragment, a Fab antibody, a secondary antibody, a ligand, and a cofactor.

229. The method of claim 228, wherein the ligand comprises a small molecule, a peptide, or a protein.

230. The method of claim 228, wherein the binding molecule is a vhh antibody.

231. The method of claim 227, wherein the linker comprises a polypeptide.

232. The method of claim 227, wherein the linker connects the N-terminus or C-terminus of the binding molecule with the 5′-end or the 3′-end of the imaging nucleic acid strand.

233. The method of claim 225, wherein the binding molecule and the imaging nucleic acid strand are connected via a sortase-mediated ligation.

234. The method of claim 233, wherein the sortase-mediated ligation comprises ligating a C-terminal LPXTG-XX sequence of the binding molecule with a GGG peptide at the 5′-end of the imaging nucleic acid strand.

235. The method of claim 225, wherein the binding molecule is connected to the imaging nucleic acid strand via a side chain of the binding molecule.

236. The method of claim 235, wherein the side chain is an arginine side chain or a cysteine side chain.

237. A method of providing a super-resolution microscopy (SRM) image of a target molecule expressed on cell surfaces of a cell sample, comprising:
a) binding a binding molecule to the target molecule, wherein the binding molecule is connected to a fluorophore and at least one cleavable moiety, and wherein the binding of the binding molecule to the target molecule brings the fluorophore in proximity to the target molecule;
b) imaging the cell sample to provide the SRM image of the target molecule; and
c) cleaving the cleavable moiety.

238. The method of claim 237, wherein cleaving the cleavable moiety displaces the fluorophore from proximity to the target molecule.

239. The method of claim 237, further comprising providing a multiplexed image of a number (n) of different target molecules expressed on the cell surfaces of the cell sample in a single step or by: repeating steps a) to c) for each of the n different target molecules to provide an SRM image of each of the n different target molecules; and combining the SRM images of the n different target molecules to provide the multiplexed image.

240. The method of claim 237, wherein imaging the cell sample comprises imaging the cell sample by stimulated emission depletion (STED) microscopy, ground state depletion (GSD) microscopy, MINFLUX microscopy, stochastic optical reconstruction microscopy (STORM), direct stochastic optical reconstruction microscopy (DSTORM), or single-molecule localization microscopy.

241. The method of claim 239, wherein n is at least 10, n is at least 20, n is at least 50, or nis at least 100.

242. The method of claim 237, wherein cleaving the cleavable moiety comprises cleaving the cleavable moiety with light.

243. The method of claim 237, wherein cleaving the cleavable moiety comprises cleaving the cleavable moiety with UV light.

244. The method of claim 237, wherein cleaving the cleavable moiety comprises cleaving the cleavable moiety via chemical cleavage.

245. The method of claim 237, wherein cleaving the cleavable moiety comprises cleaving the cleavable moiety via enzymatic cleavage.

246. The method of claim 237, wherein cleaving the cleavable moiety comprises cleaving the cleavable moiety with a nuclease.

247. The method of claim 237, wherein the cleavable moiety is located in or is part of the fluorophore.

248. The method of claim 237, further comprising fixing the cell sample prior to labeling the target molecule.

249. The method of claim 237, wherein the cell sample comprises disseminated cells.

250. The method of claim 237, wherein the cell sample comprises tissue fragments.

251. The method of claim 237, wherein the cell sample comprises a tissue sample.

252. The method of claim 251, wherein the tissue sample is fixed.

253. The method of claim 251, wherein the tissue sample is a live tissue sample.

254. The method of claim 237, wherein the cell sample comprises cultured cells.

255. The method of claim 254, wherein the cultured cells are fixed.

256. The method of claim 254, wherein the cultured cells are live.

257. The method of claim 237, wherein the method is carried out without changing buffer conditions of the cell sample.

258. The method of claim 237, wherein all of the steps of the method are carried out in a same buffer, or wherein buffer conditions and/or concentration of the binding molecule are changed at each step.

259. The method of claim 237, wherein the method is carried out in physiological buffer conditions.

260. The method of claim 237, wherein all of the steps of the method are carried out in a single buffer having a physiological pH and a physiological salt concentration.

261. The method of claim 237, wherein all of the steps of the method are carried out in a single buffer having a pH in a range of about 7.2 to about 7.5, and a sodium chloride concentration in a range of about 135 millimolar (mM) to about 145 mM.

262. The method of claim 237, wherein the method is carried out in a buffer having a sodium chloride concentration in a range of about 135 millimolar (mM) to about 145 mM.

263. The method of claim 237, wherein all of the steps of the method are carried out in phosphate-buffered saline (PBS) buffer containing 137 millimolar (mM) sodium chloride, 2.7 mM potassium chloride, 10 mM sodium phosphate dibasic, and 1.8 mM potassium phosphate monobasic at a pH of about 7.4.

264. The method of claim 237, wherein one or more steps of the method are carried out in a buffer having a salt concentration of about 50 millimolar (mM).

265. The method of claim 237, wherein the cleaving removes the fluorophore from the binding molecule.

266. The method of claim 237, further comprising washing the cell sample after the cleaving, or diluting the fluorophore with buffer after the cleaving.

267. The method of claim 237, further comprising acquiring an SRM or confocal image of the cell sample after the washing to confirm removal of the fluorophore.

268. The method of claim 237, wherein the target molecule comprises a cancer protein.

269. The method of claim 237, wherein the target molecule comprises a tumor antigen.

270. The method of claim 237, wherein the target molecule comprises a ligand.

271. The method of claim 237, wherein the target molecule comprises a lipid.

272. The method of claim 237, wherein the target molecule comprises a carbohydrate.

273. The method of claim 237, wherein the binding molecule is selected from an antibody, a vhh antibody, a single-chain variable fragment (scFv), a Fab antibody fragment, a Fab antibody, a secondary antibody, a ligand, and a cofactor.

274. The method of claim 273, wherein the ligand comprises a small molecule, a peptide, or a protein.

275. The method of claim 273, wherein the binding molecule is a vhh antibody.

276. The method of claim 237, wherein the binding molecule is connected to the cleavable moiety by a linker, optionally wherein the cleavable moiety is located in the linker.

277. The method of claim 237, wherein the binding molecule is connected to the cleavable moiety via a sortase-mediated ligation.

278. The method of claim 237, wherein the binding molecule is connected to the cleavable moiety via a side chain of the binding molecule.

279. The method of claim 278, wherein the side chain is an arginine side chain or a cysteine side chain.

280. The method of claim 237, wherein the binding molecule is connected to at least two cleavable moieties, optionally wherein the at least two cleavable moieties are located in the fluorophore, between the binding molecule and the fluorophore, or in the linker.

281. The method of claim 237, wherein the cleavable moiety is located between the binding molecule and the fluorophore or is located in or is part of the fluorophore.
